SEQUENCE LISTING (42 sequences)

SEQ ID NO: 1 - DNA, 65 bp, synthetic construct
  misc_feature 1..65: note = L-DNA oligo nucleic acid sequence
  misc_difference 17..46: note = n is a, c, g, or t
  source 1..65: mol_type = other DNA
cggatccagttacggannnnnnnnnnnnnnnnnnnnnnnnnnnnnnttcattcagtaagcttcgg

SEQ ID NO: 2 - DNA, 16 bp, synthetic construct
  misc_feature 1..16: note = L-DNA oligo nucleic acid sequence
  source 1..16: mol_type = other DNA
cggatccagttacgga

SEQ ID NO: 3 - DNA, 19 bp, synthetic construct
  misc_feature 1..19: note = L-DNA oligo nucleic acid sequence
  source 1..19: mol_type = other DNA
ccgaagcttactgaatgaa

SEQ ID NO: 4 - DNA, 20 bp, synthetic construct
  misc_feature 1..20: note = L-DNA oligo nucleic acid sequence
  source 1..20: mol_type = other DNA
aaaaaaaaaaaaaaaaaaaa

SEQ ID NO: 5 - DNA, 56 bp, synthetic construct
  misc_feature 1..56: note = L-DNA oligo nucleic acid sequence
  source 1..56: mol_type = other DNA
cgcccgccgcgccccgcgcccgtcccgccgcccccgcccgcggatccagttacgga

SEQ ID NO: 6 - DNA, 56 bp, synthetic construct; identical to SEQ ID NO: 5
  misc_feature 1..56: note = L-DNA oligo nucleic acid sequence
  modified_base 1: mod_base = OTHER; note = 5'-FAM-conjugated
  source 1..56: mol_type = other DNA

SEQ ID NO: 7 - DNA, 16 bp, synthetic construct
  misc_feature 1..16: note = L-DNA oligo nucleic acid sequence
  source 1..16: mol_type = other DNA
tggatccagttacgga

SEQ ID NO: 8 - DNA, 64 bp, synthetic construct
  misc_feature 1..64: note = L-DNA oligo nucleic acid sequence
  source 1..64: mol_type = other DNA
cggatccagttacggactgaacagaagggtggtgtggttggactgttcattcagtaagcttcgg

SEQ ID NO: 9 - DNA, 65 bp, synthetic construct
  misc_feature 1..65: note = L-DNA oligo nucleic acid sequence
  source 1..65: mol_type = other DNA
cggatccagttacggaacgcgtttcaagactaccgtgtttgttccgttcattcagtaagcttcgg

SEQ ID NO: 10 - DNA, 36 bp, synthetic construct
  misc_feature 1..36: note = L-DNA oligo nucleic acid sequence
  source 1..36: mol_type = other DNA
acggaacgcgtttcaagactaccgtgtttgttccgt

SEQ ID NO: 11 - DNA, 36 bp, synthetic construct; identical to SEQ ID NO: 10
  misc_feature 1..36: note = L-DNA oligo nucleic acid sequence
  modified_base 1: mod_base = OTHER; note = 5'-Cy5 conjugated
  source 1..36: mol_type = other DNA

SEQ ID NO: 12 - DNA, 32 bp, synthetic construct
  misc_feature 1..32: note = L-DNA oligo nucleic acid sequence
  source 1..32: mol_type = other DNA
ggaacgcgtttcaagactaccgtgtttgttcc

SEQ ID NO: 13 - DNA, 65 bp, synthetic construct
  misc_feature 1..65: note = L-DNA oligo nucleic acid sequence
  source 1..65: mol_type = other DNA
cggatccagttacggatgaacttgttgaaacccaacgggagaatcgttcattcagtaagcttcgg

SEQ ID NO: 14 - DNA, 38 bp, synthetic construct
  misc_feature 1..38: note = L-DNA oligo nucleic acid sequence
  source 1..38: mol_type = other DNA
gatgaacttgttgaaacccaacgggagaatcgttcatt

SEQ ID NO: 15 - DNA, 38 bp, synthetic construct; identical to SEQ ID NO: 14
  misc_feature 1..38: note = L-DNA oligo nucleic acid sequence
  modified_base 1: mod_base = OTHER; note = 5'-Cy5 conjugated
  source 1..38: mol_type = other DNA

SEQ ID NO: 16 - DNA, 37 bp, synthetic construct
  misc_feature 1..37: note = L-DNA oligo nucleic acid sequence
  source 1..37: mol_type = other DNA
gatgaacttgttgaaacccagcgggagatcgttcatt

SEQ ID NO: 17 - DNA, 16 bp, synthetic construct
  misc_feature 1..16: note = L-DNA oligo nucleic acid sequence
  modified_base 1: mod_base = OTHER; note = 5'-FAM-conjugated
  source 1..16: mol_type = other DNA
tccgtaactggatccg

SEQ ID NO: 18 - DNA, 15 bp, synthetic construct
  misc_feature 1..15: note = L-DNA oligo nucleic acid sequence
  modified_base 1: mod_base = OTHER; note = 5'-FAM-conjugated
  source 1..15: mol_type = other DNA
actggatccgagctg

SEQ ID NO: 19 - DNA, 13 bp, synthetic construct
  misc_feature 1..13: note = L-DNA oligo nucleic acid sequence
  modified_base 13: mod_base = OTHER; note = 3' DABCYL conjugated (4-(4-dimethyl-aminophenylazo)benzoic acid)
  source 1..13: mol_type = other DNA
acccttctgttca

SEQ ID NO: 20 - DNA, 11 bp, synthetic construct
  misc_feature 1..11: note = L-DNA oligo nucleic acid sequence
  modified_base 11: mod_base = OTHER; note = 3' DABCYL conjugated (4-(4-dimethyl-aminophenylazo)benzoic acid)
  source 1..11: mol_type = other DNA
acgcgttccgt

SEQ ID NO: 21 - DNA, 49 bp, synthetic construct
  misc_feature 1..49: note = L-DNA oligo nucleic acid sequence
  source 1..49: mol_type = other DNA
cggatccagttacggactgaacagaagggtggtgtggttggactgttca

SEQ ID NO: 22 - DNA, 52 bp, synthetic construct
  misc_feature 1..52: note = L-DNA oligo nucleic acid sequence
  source 1..52: mol_type = other DNA
cagctcggatccagttacggaacgcgtttcaagactaccgtgtttgttccgt

SEQ ID NO: 23 - DNA, 68 bp, synthetic construct
  misc_feature 1..68: note = L-DNA oligo nucleic acid sequence
  source 1..68: mol_type = other DNA
cggatccagttacggatgaacttgttgaaacccaacgggagaatcgttcattcagtaagcttcggtgg

SEQ ID NO: 24 - DNA, 16 bp, synthetic construct
  misc_feature 1..16: note = L-DNA oligo nucleic acid sequence
  source 1..16: mol_type = other DNA
cggatccagttacgga

SEQ ID NO: 25 - DNA, 18 bp, synthetic construct
  misc_feature 1..18: note = L-DNA oligo nucleic acid sequence
  source 1..18: mol_type = other DNA
ccaccgaagcttactgaa

SEQ ID NO: 26 - DNA, 20 bp, synthetic construct
  misc_feature 1..20: note = L-DNA oligo nucleic acid sequence
  source 1..20: mol_type = other DNA
aaaaaaaaaaaaaaaaaaaa

SEQ ID NO: 27 - DNA, 68 bp, synthetic construct
  misc_feature 1..68: note = L-DNA oligo nucleic acid sequence
  source 1..68: mol_type = other DNA
cggatccagttacggatgaacttgttgaaacccaacgggagcctcgttcattcagtaagcttcggtgg

SEQ ID NO: 28 - DNA, 38 bp, synthetic construct
  misc_feature 1..38: note = L-DNA oligo nucleic acid sequence
  source 1..38: mol_type = other DNA
gatgaacttgttgaaacccaacgggagcctcgttcatt

SEQ ID NO: 29 - DNA, 38 bp, synthetic construct; identical to SEQ ID NO: 28
  misc_feature 1..38: note = L-DNA oligo nucleic acid sequence
  modified_base 1: mod_base = OTHER; note = 5'-Cy5 conjugated
  source 1..38: mol_type = other DNA

SEQ ID NO: 30 - DNA, 64 bp, synthetic construct
  misc_feature 1..64: note = L-DNA oligo nucleic acid sequence
  source 1..64: mol_type = other DNA
cggatccagttacggatgaacttgttgaaacccagcgggagatcgttcattcagtaagcttcgg

SEQ ID NO: 31 - DNA, 65 bp, synthetic construct
  misc_feature 1..65: note = L-DNA oligo nucleic acid sequence
  source 1..65: mol_type = other DNA
cggatccagttacggatgaacttgttgaaacccagcgggagaatcgttcattcagtaagcttcgg

SEQ ID NO: 32 - DNA, 64 bp, synthetic construct
  misc_feature 1..64: note = L-DNA oligo nucleic acid sequence
  source 1..64: mol_type = other DNA
cggatccagttacggatgaacttgttgaaacccagcggaagatcgttcattcagtaagcttcgg

SEQ ID NO: 33 - DNA, 65 bp, synthetic construct
  misc_feature 1..65: note = L-DNA oligo nucleic acid sequence
  source 1..65: mol_type = other DNA
cggatccagttacggatgaacttgttgaaacccaacggaagaatcgttcattcagtaagcttcgg

SEQ ID NO: 34 - DNA, 64 bp, synthetic construct
  misc_feature 1..64: note = L-DNA oligo nucleic acid sequence
  source 1..64: mol_type = other DNA
cggatccagttacggatgaacttgttgaaacccaacggaagatcgttcattcagtaagcttcgg

SEQ ID NO: 35 - DNA, 64 bp, synthetic construct
  misc_feature 1..64: note = L-DNA oligo nucleic acid sequence
  source 1..64: mol_type = other DNA
cggatccagttacggatgaacttgttgaaacccaacgggagatcgttcattcagtaagcttcgg

SEQ ID NO: 36 - DNA, 65 bp, synthetic construct; identical to SEQ ID NO: 13
  misc_feature 1..65: note = L-DNA oligo nucleic acid sequence
  source 1..65: mol_type = other DNA

SEQ ID NO: 37 - DNA, 65 bp, synthetic construct
  misc_feature 1..65: note = L-DNA oligo nucleic acid sequence
  source 1..65: mol_type = other DNA
cggatccagttacggatgaacttgttgaaacccagcggaagaatcgttcattcagtaagcttcgg

SEQ ID NO: 38 - protein, 352 aa, synthetic construct
  REGION 1..352: note = synthetic Dpo4 (with S86C, N123A, S207 A, S313A and C31S.)
  REGION 1..352: note = misc_feature - D-amino acids
MIVLFVDFDYFYAQVEEVLNPSLKGKPVVVSVFSGRFEDSGAVATANYEARKFGVKAGIPIVEAKKILPNAVYLPMRKEVYQQVSCRIMNLLREYSEKIEIASIDEAYLDISDKVRDYREAYALGLEIKNKILEKEKITVTVGISKNKVFAKIAADMAKPNGIKVIDDEEVKRLIRELDIADVPGIGNITAEKLKKLGINKLVDTLAIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYAESVKLLQKILEEDERKIRRIGVRFSKFIEAIGLDKFFDT

SEQ ID NO: 39 - protein, 174 aa, synthetic construct
  REGION 1..174: note = ASFV pol X
MLTLIQGKKIVNHLRSRLAFEYNGQLIKILSKNIVAVGSLRREEKMLNDVDLLIIVPEKKLLKHVLPNIRIKGLSFSVKVCGERKCVLFIEWEKKTYQLDLFTALAEEKPYAIFHFTGPVSYLIRIRAALKKKNYKLNQYGLFKNQTLVPLKITTEKELIKELGFTYRIPKKRL

SEQ ID NO: 40 - protein, 421 aa, synthetic construct
  REGION 1..421: note = Dpo4-5m-sso7d (Dpo4-5m fused to sso7d at the C-terminus)
MIVLFVDFDYFYAQVEEVLNPSLKGKPVVVSVFSGRFEDSGAVATANYEARKFGVKAGIPIVEAKKILPNAVYLPMRKEVYQQVSCRIMNLLREYSEKIEIASIDEAYLDISDKVRDYREAYALGLEIKNKILEKEKITVTVGISKNKVFAKIAADMAKPNGIKVIDDEEVKRLIRELDIADVPGIGNITAEKLKKLGINKLVDTLAIEFDKLKGMIGEAKAKYLISLARDEYNEPIRTRVRKSIGRIVTMKRNSRNLEEIKPYLFRAIEESYYKLDKRIPKAIHVVAVTEDLDIVSRGRTFPHGISKETAYAESVKLLQKILEEDERKIRRIGVRFSKFIEAIGLDKFFDTGTGGGGATVKFKYKGEEKEVDISKIKKVWRVGKMISFTYDEGGGKTGRGAVSEKDAPKELLQMLEKQKK

SEQ ID NO: 41 - DNA, 19 bp, synthetic construct
  misc_feature 1..19: note = partial sequence of L-DNA oligo
  source 1..19: mol_type = other DNA
ccgaagcttactgaatgaa

SEQ ID NO: 42 - DNA, 18 bp, synthetic construct
  misc_feature 1..18: note = partial sequence of L-DNA oligo
  source 1..18: mol_type = other DNA
ccaccgaagcttactgaa

(19) United States
(12) Patent Application Publication
(10) Pub. No.: US 2025/0027090 A1
(43) Pub. Date: Jan. 23, 2025

(54) MIRROR-IMAGE SELECTION OF L-NUCLEIC ACID APTAMERS

(71) Applicant: Tsinghua University, Beijing (CN)

(72) Inventors: Ting ZHU, Beijing (CN); Ji CHEN, Beijing (CN)

(73) Assignee: Tsinghua University, Beijing (CN)

(21) Appl. No.: 18/786,501

(22) Filed: Jul. 28, 2024

Related U.S. Application Data
(63) Continuation of application No. PCT/IB2023/050908, filed on Feb. 2, 2023.
(60) Provisional application No. 63/311,092, filed on Feb. 17, 2022.
(60) Provisional application No. 63/306,139, filed on Feb. 3, 2022.

(51) Int. Cl. C12N 15/115; C12N 15/10; C12N 15/11

(52) U.S. Cl. CPC C12N 15/115; C12N 15/1048; C12N 15/111; C12N 2310/16; C12N 2310/531

(57) ABSTRACT

A method for screening L-nucleic acid aptamers for binding to a target molecule is disclosed. The method comprises:
(a) contacting the plurality of L-nucleic acid aptamers with the target molecule under conditions that selectively capture target-bound L-nucleic acid aptamers from the plurality of L-nucleic acid aptamers;
(b) amplifying L-nucleic acid aptamers of the target-bound L-nucleic acid aptamers to generate amplified, double-stranded L-nucleic acid oligonucleotides; and
(c) isolating amplified double stranded L-nucleic acid oligonucleotides using an electrophoresis based method, thereby screening the plurality of L-nucleic acid aptamers.

### RELATED APPLICATIONS

This application is a Continuation of PCT/IB2023/050908 having International filing date of Feb. 2, 2023, which claims the benefit of priority under 35 USC § 119 (e) of US Provisional Patent Applications Nos. 63/306,139, filed on Feb. 3, 2022 and 63/311,092, filed on Feb. 17, 2022. The contents of the above applications are all incorporated by reference as if fully set forth herein in their entirety.

### SEQUENCE LISTING STATEMENT

The XML file, entitled 99853ReplacementSequenceListing.xml, created on Sep. 19, 2024, comprising 59,941 bytes is incorporated herein by reference.

### FIELD AND BACKGROUND OF THE INVENTION

The present invention, in some embodiments thereof, relates to methods of selecting L-nucleotide aptamers and sequencing methods thereof.

Aptamers are nucleic acid polymer ligands that bind specific target molecules via tertiary interactions, selected through systematic evolution of ligands by exponential enrichment (SELEX) or in vitro selection. Natural unmodified aptamers are vulnerable to degradation by nucleases ubiquitous in vitro and in vivo, greatly limiting their practical applications as diagnostic and therapeutic tools. Although chemical modification and xeno nucleic acid (XNA) designs have been shown to enhance aptamer stability, their discovery and production require designed, specialized nucleotides, and even so, nuclease degradation of unnatural nucleic acid aptamers may not be completely avoided.

The chirally inverted L-DNA or L-RNA aptamers (mirror-image aptamers), possessing exceptional biostability both in vitro and in vivo, have been selected to bind natural target molecules. Their large-scale production can be readily implemented by automated oligo synthesizers with commercially available L-deoxynucleoside or L-ribonucleoside phosphoramidites, making them ideal for practical applications in diagnostics and therapeutics. Since the appreciation of their biochemical advantages over two decades ago, mirror-image aptamers have been selected mainly through an indirect scheme known as ‘selection-reflection’: the mirror-image version of target molecule is first chemically synthesized for the selection of D-aptamer, after which a mirror-image aptamer with the same sequence is synthesized to bind the corresponding natural target. However, the first step of chemically synthesizing the mirror-image target molecule is often problematic, especially for proteins with large sizes, extensive post-translational modifications (PTMs), and low in vitro folding efficiencies. In practice, most biologically important target molecules such as large proteins cannot be chemically synthesized and properly folded based on current technologies. As a result, only a small number of mirror-image aptamers have been discovered by selection-reflection in over two decades, all of which are targeting small molecules, short peptides, short RNAs, and small proteins, with the largest being a 110-amino acid (aa) ribonuclease from Bacillus amyloliquefaciens (barnase) at 12 kDa, whereas selections of mirror-image aptamers targeting the vast majority of biologically important, yet unsynthesizable target molecules have remained unachieved.

Background art includes U.S. patent application No. 20210332360, U.S. Pat. Nos. 11,015,178 and 10,975,370.

### SUMMARY OF THE INVENTION

According to an aspect of the present invention there is provided a method for screening a plurality of L-nucleic acid aptamers for an L-nucleic acid aptamer having a binding affinity to a target molecule, comprising:
(a) contacting the plurality of L-nucleic acid aptamers with the target molecule under conditions that selectively capture target-bound L-nucleic acid aptamers from the plurality of L-nucleic acid aptamers;
(b) amplifying L-nucleic acid aptamers of the target-bound L-nucleic acid aptamers to generate amplified, double-stranded L-nucleic acid oligonucleotides; and
(c) isolating amplified double stranded L-nucleic acid oligonucleotides using an electrophoresis based method, thereby screening the plurality of L-nucleic acid aptamers.

According to another aspect of the present invention, the kit for identifying L-nucleic acid aptamers comprising:
(i) calf intestinal phosphatase (CIP);
(ii) L-deoxyribonucleotide triphosphates (L-dNTPs) or modified L-dNTPs; and/or
(iii) a polymerase which is capable of adding one or more L-nucleotides to the 3′ end of a first L-nucleic acid.

According to another aspect of the present invention, there is provided a method of sequencing purified L-DNA molecules comprising:
(a) treating a sample comprising the purified L-DNA molecules with a phosphatase under conditions that remove 3′-monophosphates from the L-DNA molecules; and
(b) subjecting the sample to phosphorothioate sequencing, thereby sequencing purified L-DNA molecules.

According to another aspect of the present invention, there is provided an isolated thrombin-binding L-DNA aptamer comprising a sequence as set forth in SEQ ID NOs: 10, 12, 14, 16, 27 or 28 or a sequence at least 80% identical to the SEQ ID Nos: 10, 12, 14, 16, 27 or 28.

According to an embodiment of the invention, the method further comprises converting amplified double-stranded L-nucleic oligonucleotides to single stranded oligonucleotides following step (b) and prior to step (c).

According to an embodiment of the invention, the steps (a) and (b) and the step of converting are repeated at least three times prior to the isolating in order to enrich for the target-bound L-nucleic acid aptamers.

According to an embodiment of the invention, the method further comprises monitoring enrichment of the target-bound L-nucleic acid aptamers.

According to an embodiment of the invention, the monitoring is effected by an electrophoretic mobility shift assay (EMSA).

According to an embodiment of the invention, the electrophoresis based method is selected from the group consisting of Native PAGE; Denaturing PAGE; Denaturing gradient gel electrophoresis (DGGE); Constant denaturing gel electrophoresis (CDGE) and Temporal temperature gradient gel electrophoresis (TTGE).

According to an embodiment of the invention, the electrophoresis based method comprises DGGE.

According to an embodiment of the invention, the target molecule is selected from the group consisting of a peptide, a polypeptide, a small molecule, a carbohydrate and a nucleic acid molecule.

According to an embodiment of the invention, the target molecule is comprised in a cell or a tissue.

According to an embodiment of the invention, the amplifying utilizes a D-amino acid polymerase.

According to an embodiment of the invention, the D-amino acid polymerase is selected from the group consisting of D-ASFV pol X, D-Taq polymerase, D-Pfu polymerase, Sulfolobus and solfataricus P2 DNA polymerase IV (DPO4), a fusion protein comprising said DPO4 and a polymerase having an amino acid sequence at least 80% identical to the DPO4.

According to an embodiment of the invention, the polymerase has an amino acid sequence as set forth in SEQ ID NO: 38 or SEQ ID NO: 40.

According to an embodiment of the invention, the method further comprises sequencing the isolated members following step (c) so as to obtain the sequence of the L-nucleic acid aptamer having a binding affinity to the target molecule.

According to an embodiment of the invention, the sequencing is effected using a method selected from the group consisting of L-DNA chemical sequencing; L-DNA phosphorothioate sequencing; L-DNA dideoxy sequencing; L-DNA Ion Torrent sequencing; L-DNA Illumina sequencing; and L-DNA Nanopore sequencing.

According to an embodiment of the invention, the method is L-DNA phosphorothioate sequencing.

According to an embodiment of the invention, the method further comprises contacting the amplified double stranded L-nucleic acid oligonucleotides with a phosphatase prior to the sequencing.

According to an embodiment of the invention, the phosphatase comprises calf intestinal phosphatase (CIP).

According to an embodiment of the invention, each of the L-nucleic acid aptamers of the plurality of L-nucleic acid aptamers are of an identical length.

According to an embodiment of the invention, the plurality of L-nucleic acid aptamers are a library and each member of the library have an identical 5′ and 3′ nucleic acid sequence and a non-identical core sequence.

According to an embodiment of the invention, the method further comprises constructing an additional aptamer library, wherein each member of the library has an identical 5′ and 3′ nucleic acid sequence and is up to 60% randomized compared to the sequence of the isolated L-nucleic acid aptamer.

According to an embodiment of the invention, the method further comprises synthesizing the plurality of L-nucleic acid aptamers prior to step (a).

According to an embodiment of the invention, the synthesizing comprises error-prone PCR.

According to an embodiment of the invention, the error-prone PCR comprises use of an error-prone polymerase.

According to an embodiment of the invention, the core sequence comprises a random or semi-random sequence.

According to embodiments of the invention, the polymerase comprises Sulfolobus solfataricus P2 DNA polymerase IV (DPO4) or a polymerase having an amino acid sequence at least 80% identical to the DPO4.

According to embodiments of the invention, the polymerase has an amino acid sequence as set forth in SEQ ID NO: 38 or SEQ ID NO: 40.

According to embodiments of the invention, the thrombin-binding L-DNA aptamer comprising a sequence as set forth in SEQ ID Nos: 10, 14 or 28 or a sequence at least 80% identical to the SEQ ID Nos: 10, 12, 14, 16, 27 or 28.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

### IN THE DRAWINGS

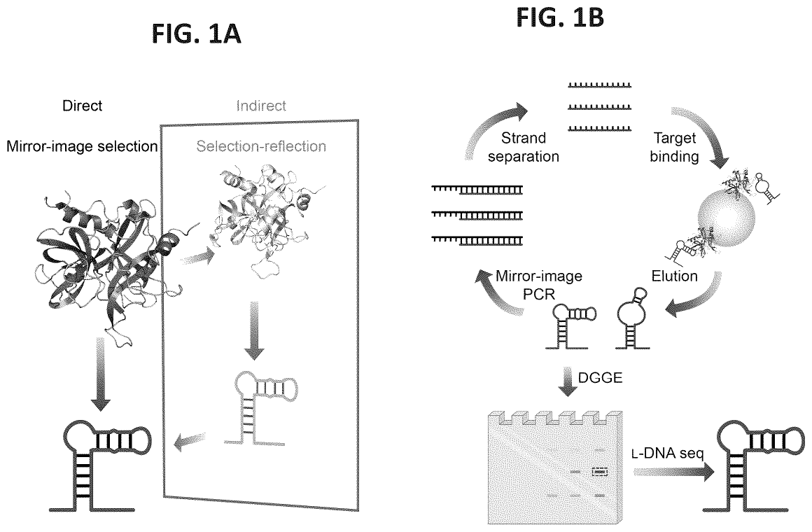

FIGS. 1A-B. Designing a mirror-image selection scheme. A, Schematic overview of the mirror-image selection of L-DNA aptamers directly from a large randomized L-DNA library (color), which bypasses the need for chemically synthesizing mirror-image target molecules as in the indirect, selection-reflection scheme (gray). PDB source: IPPB (native human thrombin). B, Schematic overview of the procedures in the mirror-image selection scheme: selection begins with a large randomized L-DNA library (e.g., with ˜1×10¹⁴ distinct L-DNA sequences in this work) to bind immobilized protein targets such as native human thrombin; the bound L-DNA is eluted and amplified by mirror-image PCR; the amplified L-DNA pool is separated into single-stranded L-DNAs for the following round; after the final round of selection, the enriched L-DNA pool is analyzed by DGGE, isolated, and sequenced with L-DNA sequencing-by-synthesis using the phosphorothioate approach.

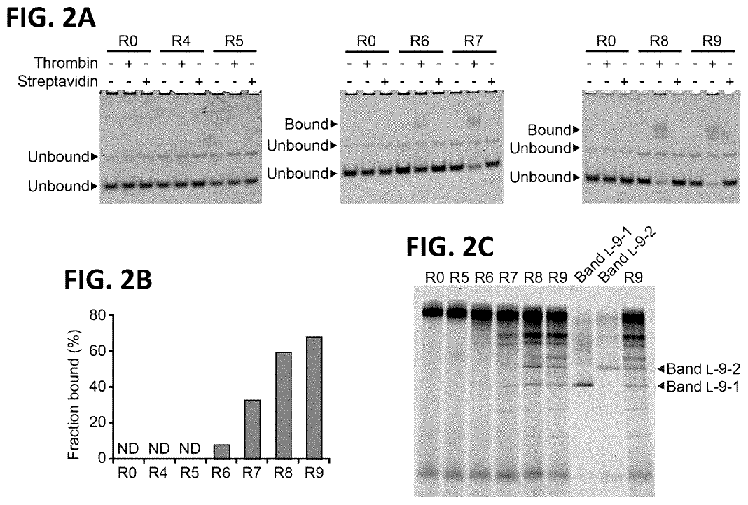

FIGS. 2A-C. Mirror-image selection of L-DNA aptamers targeting native human thrombin. A, Monitoring the progress of mirror-image selection by EMSA using 200 nM of the corresponding L-DNA pools and 1 μM native human thrombin or 1 μM streptavidin, analyzed by 8% native PAGE, and stained by SYBR Green II. B, Gel quantitation results of (A), with fraction bound determined by the ImageJ software using the band intensity of bound L-DNA pool relative to the total lane intensity. ND, (binding) not detected. C, DGGE analysis of the corresponding L-DNA pools, as well as the isolated bands L-9-1 and L-9-2, re-amplified by mirror-image PCR using D-Dpo4-5 m with L-DNA primers, analyzed by 10% denaturing PAGE in 2.1 M to 4.2 M urea and 12% to 24% formamide, and stained by SYBR-Green II.

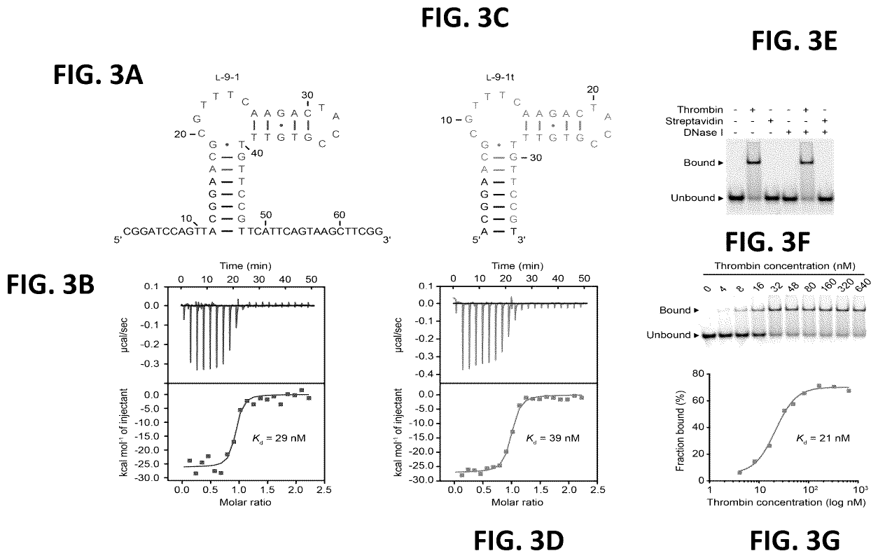

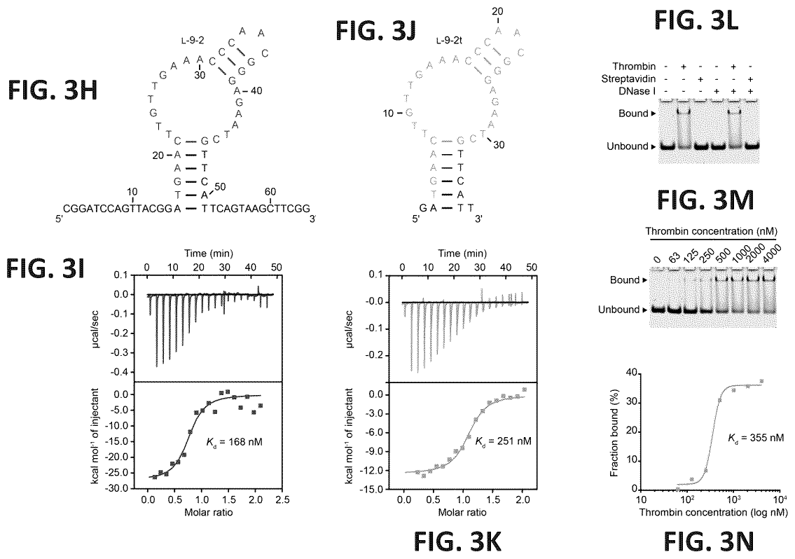

FIGS. 3A-N. Characterizing the selected L-DNA aptamers. A, Secondary structure of the L-9-1 aptamer predicted by Mfold, with nucleotides derived from the randomized region shown in blue (SEQ ID NO: 9). B, ITC analysis of the L-9-1 aptamer binding with native human thrombin, with K_{d} measured at 29 nM. C, Secondary structure of the L-9-1t (truncated version) aptamer predicted by Mfold, with nucleotides derived from the randomized region shown in cyan (SEQ ID NO: 10). D, ITC analysis of the L-9-1t aptamer binding with native human thrombin, with K_{d} measured at 39 nM. E, EMSA of 200 nM Cy5-L-9-1t aptamer binding with 1 μM native human thrombin or 1 μM streptavidin, without or with 50 units/ml DNase I, analyzed by 8% native PAGE. F, EMSA of 35 nM Cy5-L-9-1t aptamer binding with various concentrations of native human thrombin, analyzed by 8% native PAGE. G, Gel quantitation results of (f), with fraction bound determined by the ImageJ software using the band intensity of the bound Cy5-L-9-1t aptamer relative to the total lane intensity. H, Secondary structure of the L-9-2 (SEQ ID NO: 13) aptamer predicted by Mfold, with nucleotides derived from the randomized region shown in green. I, ITC analysis of the L-9-2 aptamer binding with native human thrombin, with K_{d} measured at 168 nM. J, Secondary structure of the L-9-2t (truncated version) aptamer (SEQ ID NO: 14) predicted by Mfold, with nucleotides derived from the randomized region shown in light green. K, ITC analysis of the L-9-2t aptamer binding with native human thrombin, with K_{d} measured at 251 nM. L, EMSA of 200 nM Cy5-L-9-2t aptamer binding with 1 μM native human thrombin or 1 μM streptavidin, without or with 50 units/ml DNase I, analyzed by 8% native PAGE. M, EMSA of 200 nM Cy5-L-9-2t aptamer binding with various concentrations of native human thrombin, analyzed by 10% native PAGE with 5% (v/v) glycerol. N, Gel quantitation results of (M), with fraction bound determined by the ImageJ software using the band intensity of the bound Cy5-L-9-2t aptamer relative to the total lane intensity.

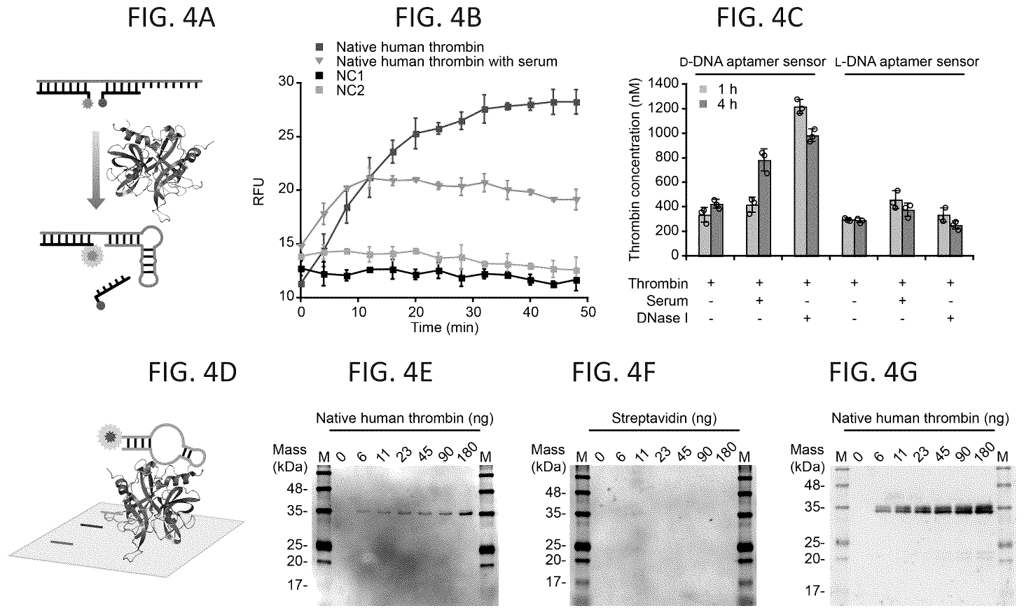

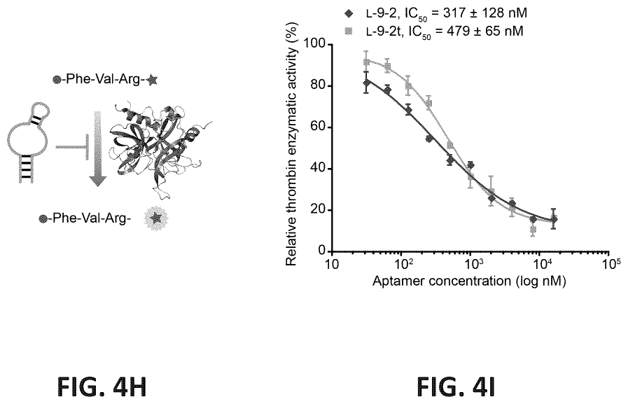

FIGS. 4A-I. Detecting and inhibiting native human thrombin with the selected L-DNA aptamers. A, Schematic overview of detecting native human thrombin using the L-DNA aptamer sensor based on the L-9-1t aptamer. B, Measured relative fluorescence for the L-DNA aptamer sensor incubated with 1 μM native human thrombin in physiological buffer alone, or physiological buffer with 10% human serum for up to 48 min, with excitation wavelength at 494 nm and emission wavelength at 518 nm, and measurements taken every 4 min. NC1, negative control in physiological buffer alone. NC2, negative control in physiological buffer with 10% human serum. RFU, relative fluorescence unit. Data are presented as mean±SD (n=3, independent measurements). C, Measured thrombin concentrations by the D- and L-DNA aptamer sensors incubated with 300 nM native human thrombin in physiological buffer alone, or in physiological buffer with 10% human serum or 50 units/ml DNase I for 1 h or 4 h. Data are presented as mean±SD (n=3, independent measurements). D, Schematic overview of detecting native human thrombin using L-DNA aptamer Western blot. E, Native human thrombin separated by 15% SDS-PAGE, transferred to a nitrocellulose membrane, incubated with 500 nM Cy5-L-13t aptamer, and scanned by the Amersham Typhoon Biomolecular Imager operated under Cy5 mode. F, Streptavidin separated by 15% SDS-PAGE, incubated with 500 nM Cy5-L-13t aptamer, and scanned by the Amersham Typhoon Biomolecular Imager operated under Cy5 mode. G, Native human thrombin separated by 15% SDS-PAGE, transferred to a nitrocellulose membrane, incubated with monoclonal primary antibody targeting native human thrombin and an Alexa Fluor 647-labelled polyclonal secondary antibody, and scanned by the Amersham Typhoon Biomolecular Imager operated under Cy5 mode. M, protein marker. H, Schematic overview of inhibiting native human thrombin enzymatic activity using the L-DNA aptamers. I, Relative thrombin enzymatic activities of the L-9-2 and L-9-2t aptamers incubated with 10 nM native human thrombin and 100 μM fluorogenic substrate benzoyl-Phe-Val-Arg-AMC in physiological buffer, with IC₅₀ measured at 317±128 nM and 479±65 nM, respectively. Data are presented as mean±SD (n=3, independent measurements).

FIGS. 5A-C. Sequencing DGGE-isolated L-DNA aptamers using the phosphorothioate approach. A, Band L-9-1 amplified by D-Dpo4-5 m with L-dNTPαSs and 5′-FAM-labelled L-DNA forward sequencing primer, cleaved by 2-iodoethanol, and analyzed by 10% denaturing PAGE. B, Band L-9-1 (SEQ ID NO: 9) amplified by D-Dpo4-5 m with L-dNTPαSs and 5′-FAM-labelled L-DNA forward sequencing primer, cleaved by 2-iodoethanol, treated by CIP, and analyzed by 10% denaturing PAGE. C, Band L-9-2 (SEQ ID NO: 13) amplified by D-Dpo4-5 m with L-dNTPαSs and 5′-FAM-labelled L-DNA forward sequencing primer, cleaved by 2-iodoethanol, treated by CIP, and analyzed by 10% denaturing PAGE. The ambiguous nucleotide positions are labeled with asterisks with the most probable substitutive nucleotides (A and G) or deletion (−) indicated.

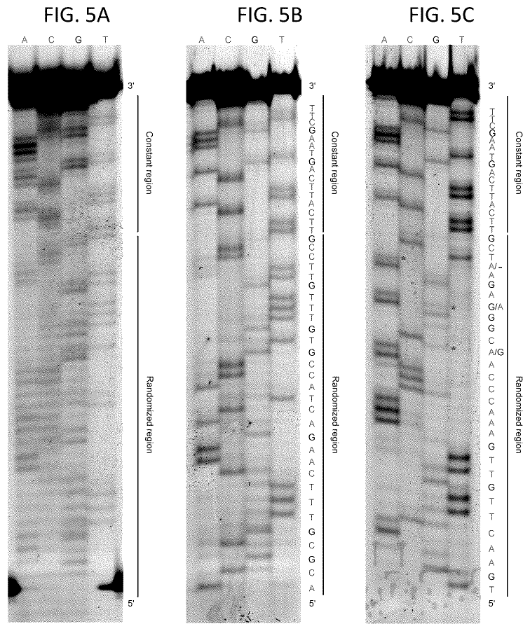

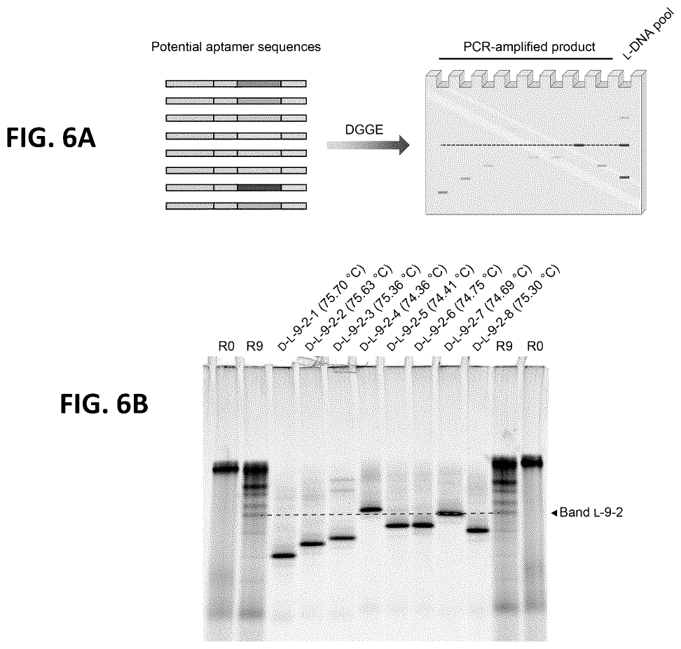

FIGS. 6A-B. Ruling out incorrect sequences from band L-9-2 sequencing results by DGGE. A, Schematic overview of ruling out incorrect sequences by DGGE, since the correct sequence(s) should co-migrate with band L-9-2 for the identical Tₘ. B, Natural versions of the eight most probable L-DNA aptamer sequences (Table 1A) in band L-9-2 (D-L-9-2-1 to D-L-9-2-8, with calculated Tₘ indicated in parentheses) amplified by natural PCR using the FastPfu Fly DNA polymerase with D-DNA primers, along with the L-DNA pools from R0 and R9, analyzed by 10% denaturing PAGE in 2.1 M to 4.2 M urea and 12% to 24% formamide, and stained by SYBR-Green II, with co-migration of D-L-9-2-7 and band L-9-2 indicated by a straight dashed blue line.

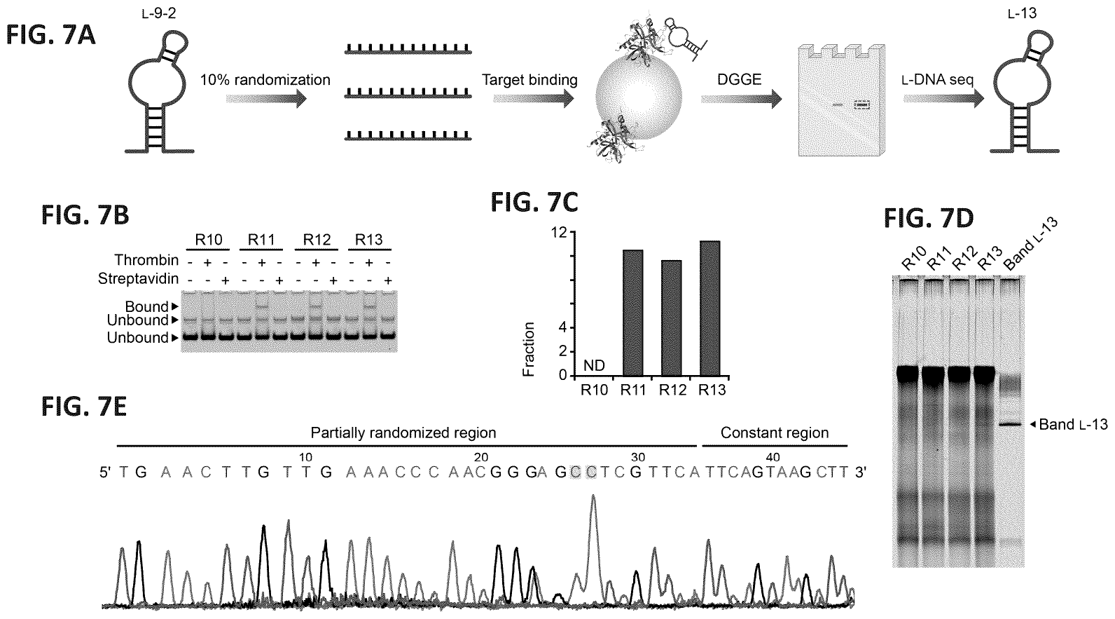

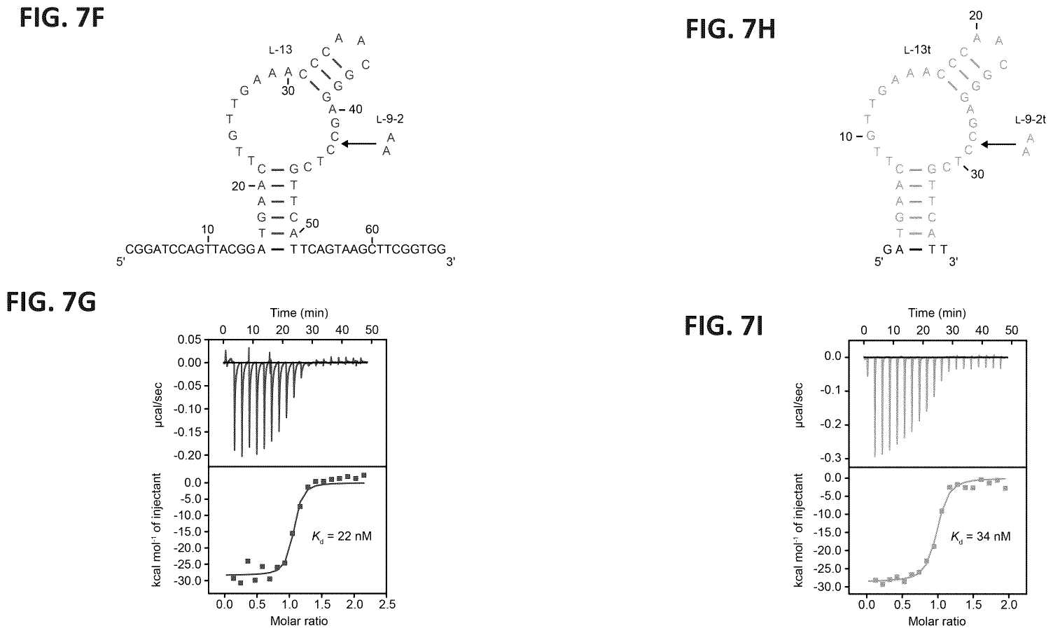

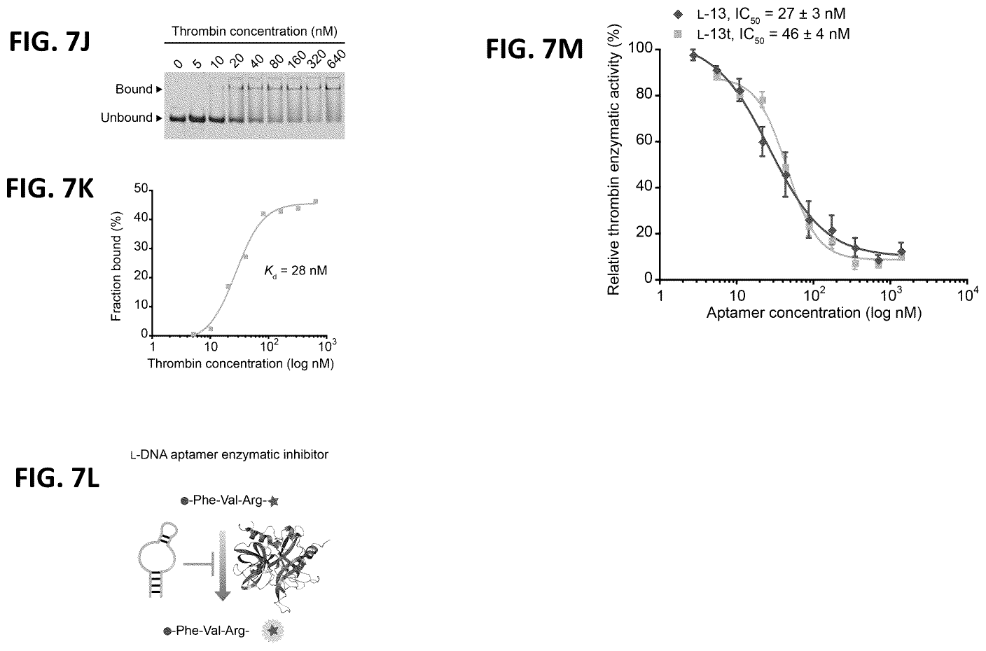

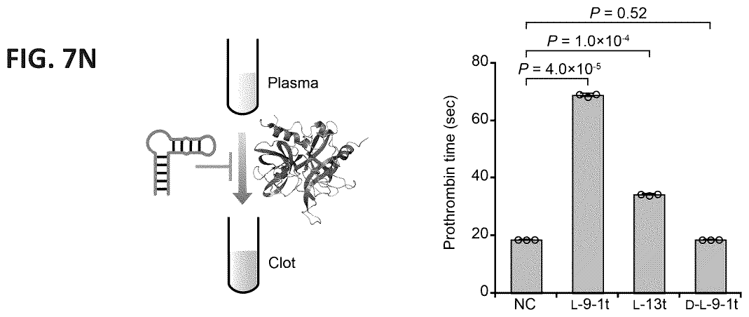

FIGS. 7A-N. Re-selection and optimization of L-DNA aptamers from a partially randomized L-DNA library. A, Schematic overview of the re-selection and optimization of L-DNA aptamers from a partially randomized L-DNA library, with partial randomization of 34 nucleotides at a frequency of 10% based on the L-9-2 aptamer. B, Monitoring the progress of mirror-image selection by EMSA using 200 nM of the corresponding L-DNA pools and 1 μM native human thrombin or 1 μM streptavidin, analyzed by 8% native PAGE, and stained by SYBR Green II. C, Gel quantitation results of (B), with the fraction bound determined by the ImageJ software using the band intensity of bound L-DNA pool relative to the total lane intensity. ND, (binding) not detected. D, DGGE analysis of the corresponding L-DNA pools, as well as the isolated band L-13, re-amplified by mirror-image PCR using D-Dpo4-5 m with L-DNA primers, analyzed by 10% denaturing PAGE in 2.1 M to 4.2 M urea and 12% to 24% formamide, and stained by SYBR-Green II. E, Sequencing chromatogram of band L-13 by D-Dpo4-5 m with L-dNTPαSs and 5′-FAM-labelled L-DNA sequencing primer after natural CIP treatment (with the two mutations highlighted in yellow). F, Secondary structure of the L-13 aptamer (SEQ ID NO: 27) predicted by Mfold, with nucleotides derived from the re-selection shown in red and the two mutations (adenosines to cytidines) indicated. G, ITC analysis of the L-13 aptamer binding with native human thrombin, with K_{d} measured at 22 nM. H, Secondary structure of the L-13t (truncated version) aptamer (SEQ ID NO: 28) predicted by Mfold, with nucleotides derived from the re-selection shown in pink and the two mutations (adenosines to cytidines) indicated. I, ITC analysis of the L-13t aptamer binding with native human thrombin, with K_{d} measured at 34 nM. J, EMSA of 35 nM Cy5-L-13t aptamer binding with various concentrations of native human thrombin, analyzed by 8% native PAGE. K, Gel quantitation results of (J), with fraction bound determined by the ImageJ software using the band intensity of the bound Cy5-L-13t aptamer relative to the total lane intensity. L, Schematic overview of inhibiting native human thrombin enzymatic activity using the re-selected L-DNA aptamers. M, Relative thrombin enzymatic activities of the L-13 and L-13t aptamers incubated with 10 nM native human thrombin and 100 μM fluorogenic substrate benzoyl-Phe-Val-Arg-AMC in physiological buffer, with IC₅₀ measured at 27±3 nM and 46±4 nM, respectively. Data are presented as mean±SD (n=3, independent measurements). N, Schematic overview of anticoagulation using the L-DNA aptamers. Prothrombin time measured with 2.5 μM L-9-1t, L-13t, and the natural version of the L-9-1t (D-L-9-1t) aptamers in the presence of 50% (v/v) human plasma. NC, negative control with physiological buffer alone. Data are presented as mean±SD (n=3, independent measurements, two-tailed unpaired student t test).

### DESCRIPTION OF SPECIFIC EMBODIMENTS OF THE INVENTION

The present invention, in some embodiments thereof, relates to methods of selecting L-nucleotide aptamers and sequencing methods thereof.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Mirror-image aptamers made from chirally inverted nucleic acids are nuclease-resistant and exceptionally biostable. Despite their diagnostic and therapeutic potential, only a small number of mirror-image aptamers have been selected by indirect selection schemes such as ‘selection-reflection’, mainly because the vast majority of biologically important target molecules such as large proteins cannot be chemically synthesized and properly folded. The present inventors have now developed a ‘mirror-image selection’ scheme for discovering L-DNA aptamers, directly selected from a large randomized L-DNA library, using mirror-image molecular tools (see FIG. 1A). The present inventors performed iterative rounds of enrichment and D-amino acid polymerase chain reaction (PCR) amplification for L-DNA sequences that bind native human thrombin, in conjunction with denaturing gradient gel electrophoresis (DGGE) to isolate and L-DNA sequencing-by-synthesis to determine the enriched L-DNA aptamer sequences, identifying several high-affinity thrombin-binding L-DNA aptamers (as illustrated in FIG. 1B).

Whilst further reducing the present invention to practice, the present inventors designed sensors and inhibitors based on the selected L-DNA aptamers, which functioned in physiologically relevant nuclease-rich environments, even in the presence of human serum that rapidly degraded D-DNA aptamers (as demonstrated in FIGS. 4B-C, 4H-I, and 7N).

The realization of a direct, mirror-image selection scheme for L-DNA aptamers greatly expands the applications of mirror-image biology systems, towards fully unlocking the potential of mirror-image aptamers as biostable biosensors, therapeutic agents, as well as basic research tools. The biostability of the L-DNA pools and the mirror-image molecular tools will also make the system entirely resistant to degradation by contaminating nucleases and proteases, especially for low-purity target molecules and cell- and tissue-based selections.

Thus, according to a first aspect of the present invention, there is provided a method for screening a plurality of L-nucleic acid aptamers for an L-nucleic acid aptamer having a binding affinity to a target molecule, comprising:
(a) contacting the plurality of L-nucleic acid aptamers with the target molecule under conditions that selectively capture target-bound L-nucleic acid aptamers from the plurality of L-nucleic acid aptamers;
(b) amplifying L-nucleic acid aptamers of the target-bound L-nucleic acid aptamers to generate amplified, double-stranded L-nucleic acid oligonucleotides;
(c) isolating amplified double stranded L-nucleic acid oligonucleotides using an electrophoresis based method, thereby screening the plurality of L-nucleic acid aptamers.

As used herein, the term “aptamer” refers to a nucleic acid molecule which shows a specific binding affinity to a target molecule, wherein such target is other than a polynucleotide that binds to the aptamer sequence through a mechanism which predominantly depends on Watson/Crick base pairing.

The phrase “L-nucleic acid aptamer” refers to an aptamer that comprises at least one L-deoxyribonucleotide or at least one L-ribonucleotide. According to a particular embodiment, at least 50% of the nucleotides of the L-nucleic acid aptamer are L-nucleotides. In still another embodiment, all the nucleotides of the L-nucleic acid aptamer are L-nucleotides. Here, it is also intended that instead of deoxyribose or ribose other sugars may form the sugar component of the nucleotide. Furthermore, the use of nucleotides with further modifications at position 2′ is comprised, such as NH₂, OMe, OBt, OAlkyl, NHAlkyl and the use of natural and non-natural nucleobases, as for example isocytidine, isoguanosine.

The L-nucleic acid aptamer may be double or single stranded. Typically, it is a single stranded L-nucleic acid, which may, however, form defined secondary structures and thus tertiary structures also, due to its primary sequence. In the secondary structure a multitude of L-nucleic acids has double stranded sections.

The target molecule may be a peptide (e.g., a naturally occurring or a synthetic peptide), a protein (or a portion thereof), a sugar (e.g., a monosaccharide or a polysaccharide), a lipid, a small molecule (e.g., less than 1500 daltons), a mixture of cellular membrane fragments, or a microorganism. In some embodiments, a target molecule excludes any nucleotide or polynucleotide molecules.

According to a particular embodiment, the target molecule is a protein (or portion thereof). The binding affinity (Kd) of the aptamer to the target molecule is preferably less than 2000 nM, less than 1000 nM, less than 750 nM, less than 500 nM, less than 250 nM, less than 100 nM and even less than 50 nM, as measured by EMSA (in the absence of serum).

As mentioned, the aptamer selected according to the methods described herein, specifically (or selectively) binds to its target i.e. the aptamer binds to the target molecule with at least 10, 20 fold or even 50 fold higher affinity than to a non-target molecule of the same type. Thus, for example if the aptamer selectively binds to a protein (for example human thrombin), it binds to the thrombin with at least 10 fold higher affinity than to a protein of similar size (for example bovine thrombin).

The method for selecting candidate aptamers starts with contacting a plurality of L-nucleic acid aptamer candidates with the target molecule under conditions that selectively capture target-bound L-nucleic acid aptamers from the plurality of L-nucleic acid aptamer candidates.

### Synthesis of L-Nucleic Acid Aptamer Candidates

The plurality of L-nucleic acid aptamer candidates comprise any number of candidates, for example at least 10, at least 100, at least 1000, each having a non-identical sequence. The candidate L-nucleic acid aptamers may all be of an identical length or may be of different lengths. Exemplary lengths of L-nucleic acid aptamers is between 20-500 nucleotides in length, 20-400 nucleotides in length, 20-300 nucleotides in length, 20-200 nucleotides in length and between 20-100 nucleotides in length.

Chemical synthesis of L-nucleic acid aptamers can be carried out by solid phase synthesis using L-DNA phosphoramidite chemistry, as known in the art. The candidate L-nucleic acid aptamers may be purified following synthesis using methods known in the art including, but not limited to native polyacrylamide gel electrophoresis so as to remove aggregation-prone L-nucleic acid aptamers.

In one embodiment, the plurality of L-nucleic acid aptamer candidates are members of a library, wherein each member of the library has an identical 5′ and 3′ nucleic acid sequence and a non-identical (e.g. random) core sequence. The core sequence may be between 10-100 nucleotides in length, between 10-80 nucleotides in length, between 10-70 nucleotides in length, between 10-60 nucleotides in length, between 10-50 nucleotides in length, between 10-40 nucleotides in length, between 10-30 nucleotides in length.

The preparation of such combinatorial libraries is described, for example, in Conrad, R. C., Giver, L., Tian, Y. and Ellington, A. D., 1996, Methods Enzymol., Vol 267, 336-367.

In order to efficiently increase members of a library having identical 5′ and 3′ sequences, the chemically synthesized aptamers may be amplified by error-prone PCR, whereby the 5′ and 3′ ends (being primer binding sites) are kept constant by virtue of the primers using during the PCR reaction, and the core is subject to error prone PCR. In one embodiment, the error-prone PCR utilizes an error prone polymerase (e.g. Dpo4 or Taq DNA polymerase). In another embodiment, a high-fidelity polymerase (e.g. Pfu DNA polymerase) is used and the amplification conditions are selected that promote insertion of errors (e.g. addition of Mn²⁺).

It will be appreciated that the amount of variation in the L-DNA candidate pool may be controlled during chemical synthesis by doping wild-type nucleotides with each of the other three L-DNA nucleotides.

In addition, an RNA library may, in principle, be generated from double stranded DNA, if a T7 promoter has been included previously, also by a suitable DNA dependent RNA polymerase, e.g. T7 RNA polymerase. Aided by the methods described, it is possible to generate libraries of 1015 and more DNA or RNA molecules. Every molecule from this library has a different sequence and thus a different three-dimensional structure.

### Capture Target-Bound L-Nucleic Acid Aptamers

In order to separate between L-nucleic acid aptamers that bind with a high affinity to the target and L-nucleic acid aptamers that bind with a lower affinity to the target, the target may be used as a bait to capture the target-binding aptamers. This serves to enrich the pool for target binding L-nucleic acid aptamers.

In order to capture the target-binding aptamers, the target molecule may be immobilized on to a solid support. Exemplary solid supports include, but are not limited to laminated graphenes, carbon nanotubes, fullerenes and particles. Examples of materials that can be used to fabricate the particles include, but are not limited to silica beads, polystyrene beads, latex beads, and metal colloids may be included. According to a particular embodiment, the particles are magnetic particles. The target molecule may be immobilized on the solid phase support surface by a hydrophobic interaction, an electrostatic interaction, a covalent bond, a coordination bond, or a noncovalent intermolecular action (such as biotin-streptavidin).

In other embodiments, the target molecule may be attached to a readable label, e.g., a fluorescent label, such that the signal from the aptamer-bound target molecule may be read and recorded using, e.g., FACS. In other embodiments, the target molecule may not contain a readable label. In such scenarios, the aptamers in a library to be screened may have certain scaffolds (e.g., hairpin scaffold and displacement strand) that change their structures upon aptamer binding to the target molecule. The conformational change induced by target molecule binding may in turn generate a readable signal (for example due to FRET interactions) to be recorded.

It will be appreciated that prior to capturing L-nucleic acid aptamers that bind the target molecule the candidate pool may be pre-enriched by at least one round of negative selection (i.e.

depletion of the candidate pool of sequences that bind non-specifically to non-targets). For example a selection may be carried out against bead-immobilized human serum may be performed to reduce the number of aptamers that bind nonspecifically to non-targets.

### Amplifying L-Nucleic Acid Aptamers of the Target-Bound L-Nucleic Acid Aptamers

Once target bound aptamers are separated from the non-target bound aptamers, they may be amplified using a mirror-image PCR reaction.

The term “mirror-image” as used herein, refers to an isomer that is in a mirror-image relationship with the natural material in chirality.

The phrase “mirror-image PCR reaction” refers to a polymerase chain reaction which incorporates L-nucleotides into the amplified sequence.

The mirror image PCR reaction typically used a mirror-image polymerase which is a D-amino acid polymerase that is in a mirror-image relationship with a native polymerase (ie, an L-form polymerase). The term “mirror-image polymerase” is used interchangeably with “D-form polymerase” or “D amino acid polymerase.” For example, “D-Dpo4” refers to D-form Dpo4 polymerase which is in a mirror-image relationship with the native L-form Dpo4 polymerase.

The polymerase particularly suitable for the present invention includes D-ASFV pol X, D-Dpo4, D-Taq polymerase, D-Pfu polymerase and functional variants thereof.

Dpo4 (Sulfolobus solfataricus P2 DNA polymerase IV) is a thermostable polymerase which can also synthesize DNA at 37° C. Its mismatch rate is between 8×10⁻³ to 3×10⁻⁴. It is a polymerase that can replace Taq for multi-cycle PCR reaction. Its amino acid sequence length is within the reach of current chemical synthesis techniques.

Taq polymerase is a thermostable polymerase which remains active at DNA denaturation temperatures. The optimum temperature for Taq is between 75° C. and 80° C. and the half-life at 92.5° C. is about 2 hours.

Pfu polymerase is found in Pyrococcus furiosus, and its function in microorganisms is to replicate DNA during cell division. It is superior to Taq in that it has 3′-5′ exonuclease activity and can cleave the mis-added nucleotides on the extended strand during DNA synthesis. The mismatch rate of commercial Pfu is around 1 in 1.3 million.

The term “functional variant” as used herein refers to a variant comprising substitution, deletion or addition of one or more (for example, 1-5, 1-10 or 1-15, in particular, such as 1, 2, 3, 4, 5, 6, 7, 8, 9, 10, 11, 12, 15 or even more) amino acids in the amino acid sequence of a wild-type enzyme, and the variant substantially retains the biology of the wild-type enzyme. For example, 50%, 60%, 70%, 80% or 90% or more of the biological activity of the wild type enzyme is retained. The “functional variant” may be a naturally occurring variant, or an artificial variant, such as a variant obtained by site-directed mutagenesis, or a variant produced by a genetic recombination method.

In a preferred embodiment of the present invention, the mirror-image nucleic acid polymerase may comprise an affinity tag to facilitate purification and reuse of the protein, such as a polyhistidine tag (His-Tag or His tag), a polyarginine tag, a glutathione-S-transferase tag, and the like.

A particular functional variant of Dpo4 protein is Dpo4-5 m, which comprises amino acid mutations at 5 positions. In one embodiment, the Dpo4 protein comprises at least one, two, three, four or each of the following mutations: C31S, S86C, NI23A, S207A and S313A.

The amino acid sequence of Dpo4-5 m polymerase may comprise a sequence as set forth in SEQ ID NO: 38.

In another embodiment, the Dpo4-5 m polymerase comprises am Sso7d domain fused to the C-terminus of Dpo4-5 m (an exemplary sequence being set forth in SEQ ID NO: 40).

In one embodiment, the mirror-image PCR is performed in a buffer of 50 mM Tris-HCl, pH 7.5, 20 mM MgCl₂, 1 mM DTT, and 50 mM KCl.

The present invention also provides D-ASFV pol X, the sequence of which is set forth in SEQ ID NO: 39, wherein except for glycine which is not chiral, all other amino acids are D-form amino acids.

In some embodiments, the mirror-image nucleic acid, the mirror-image nucleic acid template, the mirror-image nucleic acid primer, and the mirror-image dNTPs/rNTPs are in L-form, and the mirror-image nucleic acid polymerase is in D-form.

Herein, the nucleic acid replication reaction may be carried out in only one cycle or in multiple cycles. This may be determined by persons skilled in the art according to actual needs.

The term “multiple” as used herein refers to at least two. For example, “multiple cycles” refers to 2 or more cycles, such as 3, 4, or 10 cycles.

The term “replication” as used herein includes obtaining one or more copies of a target DNA in the presence of a DNA template and dNTPs; and also obtaining one or more copies of a target RNA in the presence of a DNA template and rNTPs (this process may also be known as RNA “transcription”). In the process of nucleic acid replication, the template and the primer are usually DNA. If the target nucleic acid is DNA, dNTPs should be added to the reaction system; if the target nucleic acid is RNA, rNTPs should be added to the reaction system.

In a particularly preferred embodiment, the reaction is carried out in a buffer of 50 mM Tris-HCl, pH 7.5, 20 mM MgCl₂, 1 mM DTT, and 50 mM KCl.

It will be appreciated that if the L-nucleic acid aptamer is an RNA aptamer, a reverse transcription reaction should be carried out prior to amplification step by polymerase chain reaction. A library enriched after a first round of selection may be used for a renewed round of selection, such that the molecules enriched in the first round of selection have a chance to prevail again by selection and amplification and go into a further round of selection with even more daughter molecules. An enriched pool emerges this way, whose members are then separated using an electrophoresis based method as further described below.

For multiple rounds of selection, the amplified aptamer sequence (which is double-stranded) is converted into a single-stranded nucleic acid sequence prior to addition of the target.

Methods of obtaining single stranded nucleic acids are known in the art and the present invention contemplates use of any of these methods. In one particular embodiment, a spacer is used to interrupt the reverse primer (e.g. Sp18 spacer), so that the PCR product contains two strands of unequal lengths. Denaturing PAGE can then be used to separate the two strands (see Examples section herein below).

In another embodiment a binding moiety is used to modify one of the reverse primers (e.g. biotin) and the double stranded DNA is captured by an agent that binds specifically to the binding moiety (e.g. streptavidin coated beads). The strand without the binding moiety may be eluted using NaOH, whereas the strand with the binding moiety remains attached to the agent.

The present invention contemplates at least 3 rounds of selection, amplification and conversion to a single-stranded aptamer, at least 4 rounds of selection, amplification and conversion to a single-stranded aptamer, at least 5 rounds of selection, amplification and conversion to a single-stranded aptamer, at least 6 rounds of selection, amplification and conversion to a single-stranded aptamer. In one embodiment, no more than 10 rounds of selection, amplification and conversion to a single-stranded aptamer are carried out. In still another embodiment, no more than 15 rounds of selection, amplification and conversion to a single-stranded aptamer are carried out.

The enrichment of the L-nucleic acid aptamer pool for those that bind the target may be monitored using methods known in the art. Such methods include electromobility shift assay (EMSA).

As mentioned following sufficient enrichment of the L-nulceic acid aptamer pool, the resultant aptamers are further purified using an electrophoresis based method, as further described below.

### Isolating Amplified L-Nucleic Acid Oligonucleotides

Electrophoresis based methods for isolating aptamers which bind to the target include but are not limited to Native PAGE; Denaturing PAGE; Denaturing gradient gel electrophoresis (DGGE); Constant denaturing gel electrophoresis (CDGE), capillary electrophoresis and temporal temperature gradient gel electrophoresis (TTGE).

According to a particular embodiment, the electrophoresis based method which separates the candidate target-binding aptamer is DGGE.

Denaturing/Temperature Gradient Gel Electrophoresis (DGGE/TGGE): This is a method which relies on detecting changes in electrophoretic mobility in response to minor sequence changes. One of these methods, termed “Denaturing Gradient Gel Electrophoresis” (DGGE) is based on the observation that slightly different sequences will display different patterns of local melting when electrophoretically resolved on a gradient gel. In this manner, variants can be distinguished, as differences in melting properties of homoduplexes versus heteroduplexes differing in a single nucleotide can detect the presence of SNPs in the target sequences because of the corresponding changes in their electrophoretic mobilities. The fragments to be analyzed, usually PCR products, are “clamped” at one end by a long stretch of G-C base pairs (30-80) to allow complete denaturation of the sequence of interest without complete dissociation of the strands. The attachment of a GC “clamp” to the DNA fragments increases the fraction of mutations that can be recognized by DGGE (Abrams et al., Genomics 7:463-475, 1990). Attaching a GC clamp to one primer is critical to ensure that the amplified sequence has a low dissociation temperature (Sheffield et al., Proc. Natl. Acad. Sci., 86:232-236, 1989; and Lerman and Silverstein, Meth. Enzymol., 155:482-501, 1987). Modifications of the technique have been developed, using temperature gradients (Wartell et al., Nucl. Acids Res., 18:2699-2701, 1990), and the method can be also applied to RNA: RNA duplexes (Smith et al., Genomics 3:217-223, 1988).

Limitations on the utility of DGGE include the requirement that the denaturing conditions must be optimized for each type of DNA to be tested. Furthermore, the method requires specialized equipment to prepare the gels and maintain the needed high temperatures during electrophoresis. The expense associated with the synthesis of the clamping tail on one oligonucleotide for each sequence to be tested is also a major consideration. In addition, long running times are required for DGGE. The long running time of DGGE was shortened in a modification of DGGE called constant denaturant gel electrophoresis (CDGE) (Borrensen et al., Proc. Natl. Acad. Sci. USA 88:8405, 1991). CDGE requires that gels be performed under different denaturant conditions in order to reach high efficiency for the detection of SNPs.

A technique analogous to DGGE, termed temperature gradient gel electrophoresis (TGGE), uses a thermal gradient rather than a chemical denaturant gradient (Scholz, et al., Hum. Mol. Genet. 2:2155, 1993). TGGE requires the use of specialized equipment which can generate a temperature gradient perpendicularly oriented relative to the electrical field. TGGE can detect mutations in relatively small fragments of DNA therefore scanning of large gene segments requires the use of multiple PCR products prior to running the gel.

Following separation by electrophoresis, the isolated L-DNA aptamer may be sequenced using methods known in the art.

Exemplary methods for sequencing L-DNA aptamers include but are not limited to L-DNA chemical sequencing; L-DNA phosphorothioate sequencing; L-DNA dideoxy sequencing; L-DNA Ion Torrent sequencing; L-DNA Illumina sequencing; and L-DNA Nanopore sequencing.

High throughput methods can comprise techniques to rapidly sequence a large number of nucleic acids, including next generation techniques such as Massively parallel signature sequencing (MPSS; Polony sequencing; 454 pyrosequencing; Illumina (Solexa) sequencing; SOLID sequencing; Ion Torrent semiconductor sequencing; DNA nanoball sequencing; Heliscope single molecule sequencing; Single molecule real time (SMRT) sequencing, or other methods such as Nanopore DNA sequencing; Tunneling currents DNA sequencing; Sequencing by hybridization; Sequencing with mass spectrometry; Microfluidic Sanger sequencing; Microscopy-based techniques; RNAP sequencing; In vitro virus high-throughput sequencing.

The isolated L-nucleotide aptamers may be subjected to automated dideoxy terminator sequencing reactions using a dye-terminator (unlabeled primer and labeled di-deoxy nucleotides) or a dye-primer (labeled primers and unlabeled di-deoxy nucleotides) cycle sequencing protocols. For the dye-terminator reaction, a PCR reaction is performed using unlabeled PCR primers followed by a sequencing reaction in the presence of one of the primers, deoxynucleotides and labeled di-deoxy nucleotide mix. For the dye-primer reaction, a PCR reaction is performed using PCR primers conjugated to a universal or reverse primers (one at each direction) followed by a sequencing reaction in the presence of four separate mixes (correspond to the A, G, C, T nucleotides) each containing a labeled primer specific the universal or reverse sequence and the corresponding unlabeled di-deoxy nucleotides.

Pyrosequencing™ analysis (Pyrosequencing, Inc. Westborough, MA, USA): This technique is based on the hybridization of a sequencing primer to a single stranded, PCR-amplified, DNA template in the presence of DNA polymerase, ATP sulfurylase, luciferase and apyrase enzymes and the adenosine 5′ phosphosulfate (APS) and luciferin substrates. In the second step the first of four deoxynucleotide triphosphates (dNTP) is added to the reaction and the DNA polymerase catalyzes the incorporation of the deoxynucleotide triphosphate into the DNA strand, if it is complementary to the base in the template strand. Each incorporation event is accompanied by release of pyrophosphate (PPi) in a quantity equimolar to the amount of incorporated nucleotide. In the last step the ATP sulfurylase quantitatively converts PPi to ATP in the presence of adenosine 5′ phosphosulfate. This ATP drives the luciferase-mediated conversion of luciferin to oxyluciferin that generates visible light in amounts that are proportional to the amount of ATP. The light produced in the luciferase-catalyzed reaction is detected by a charge coupled device (CCD) camera and seen as a peak in a pyrogram™. Each light signal is proportional to the number of nucleotides incorporated.

Phosphorothioate sequencing: Phosphorothioate sequencing may be carried out by performing a mirror-image PCR reaction (e.g. using D-Dpo4-5 m) in which one of the L-dNTPs is replaced by the corresponding L-dNTPαS. The product is was mixed with a solution containing 2-iodoethanol. In one embodiment, the 3′-monophosphate is first removed from the 2-iodoethanol-cleaved DNA fragments using a phosphatase (e.g. calf intestinal phosphatase-CIP) before running on a denaturing sequencing gel. More information on phosphorothioate sequencing may be found in Fan, C., et al Nat. Biotechnol. 39:1548-1555 (2021), the contents of which is incorporated herein by reference.

Once the sequence is obtained, the L-DNA aptamer may be chemically synthesized and its binding activity for its corresponding target may be verified.

Once a lead candidate sequence is obtained, it may be used as a starting point to create a new library, and therefore identify additional candidates with improved affinity/specificity. The lead sequence may be partially randomized (e.g. 1-60% randomized. In one embodiment, the lead candidate sequence is mutated at 10% randomization (˜3.3% for each base other than the original base), Thus the doping rate may be between 1% to 60%.

Agents used to isolate the L-DNA aptamers of the invention may, if desired, be presented in a kit. The kit may be accompanied by instructions for use.

According to a specific embodiment, the kit comprises:
(i) calf intestinal phosphatase (CIP);
(ii) L-deoxyribonucleotide triphosphates (L-dNTPs) or modified L-dNTPs; and/or
(iii) (iii) a polymerase which is capable of adding one or more L-nucleotides to the 3′ end of a first L-nucleic acid.
   Modified L-dNTPs include L-deoxynucleoside α-thiotriphosphate,

The aptamers of the invention can be used in various methods to assess presence or level of biomarkers in a biological sample, e.g., biological entities of interest such as proteins, sugars, cells or microvesicles. The aptamer functions as a binding agent to assess presence or level of the cognate target molecule. Therefore, in various embodiments of the invention directed to diagnostics, prognostics or theranostics, one or more aptamers of the invention are configured in a ligand-target based assay, where one or more aptamer of the invention is contacted with a selected biological sample, where the or more aptamer associates with or binds to its target molecules. Aptamers of the invention are used to identify candidate biosignatures based on the biological samples assessed and biomarkers detected.

The L-nucleic acid aptamers uncovered by methods of the present invention include those having a nucleic acid sequence as set forth in SEQ ID NOs: 10, 12, 14, 16, 27 or 28. In one embodiment, the L-nucleic acid aptamer is at least 80%, 81%, 82%, 83%, 84%, 85%, 86%, 87%, 88%, 89%, 90%, 91%, 92%, 93%, 94%, 95%, 96%, 97%, 98%, 99% identical to SEQ ID Nos: 10, 14 or 28. In another embodiment the L-nucleic acid aptamers have a sequence as set forth in SEQ ID Nos: 10, 12, 14, 16, 27 or 28 wherein up to 10 nucleotides of the sequence are mutated, wherein the position of the mutation is a single stranded region of the aptamer (as predicted by computational analyses such as Mfold). In one embodiment, the single stranded region is the one shown in FIGS. 3A, 3C, 3H, 3J, 7F or 7H.

The aptamers described herein may be attached to a detectable moiety or a label.

Appropriate labels include without limitation a magnetic label, a fluorescent moiety, an enzyme, a chemiluminescent probe, a metal particle, a non-metal colloidal particle, a polymeric dye particle, a pigment molecule, a pigment particle, an electrochemically active species, semiconductor nanocrystal or other nanoparticles including quantum dots or gold particles, fluorophores, quantum dots, or radioactive labels. Protein labels include green fluorescent protein (GFP) and variants thereof (e.g., cyan fluorescent protein and yellow fluorescent protein); and luminescent proteins such as luciferase, as described below. Radioactive labels include without limitation radioisotopes (radionuclides), such as 3H, 11C, 14C, 18F, 32P, 35S, 64Cu, 68Ga, 86Y, 99Tc, 111 In, 1231, 1241, 1251, 1311, 133Xe, 77Lu, 211At, or 213Bi. Fluorescent labels include without limitation a rare earth chelate (e.g., europium chelate), rhodamine; fluorescein types including without limitation FITC, 5-carboxyfluorescein, 6-carboxy fluorescein; a rhodamine type including without limitation TAMRA; dansyl; Lissamine; cyanines; phycoerythrins; Texas Red; Cy3, Cy5, dapoxyl, NBD, Cascade Yellow, dansyl, PyMPO, pyrene, 7-diethylaminocoumarin-3-carboxylic acid and other coumarin derivatives, Marina Blue™, Pacific Blue™, Cascade Blue™, 2-anthracenesulfonyl, PyMPO, 3,4,9,10-perylene-tetracarboxylic acid, 2,7-difluorofluorescein (Oregon Green™ 488-X), 5-carboxyfluorescein, Texas Red™-X, Alexa Fluor 430, 5-carboxytetramethylrhodamine (5-TAMRA), 6-carboxytetramethyrhodamine (6-TAMRA), BODIPY FL, bimane, and Alexa Fluor 350, 405, 488, 500, 514, 532, 546, 555, 568, 594, 610, 633, 647, 660, 680, 700, and 750, and derivatives thereof, among many others. See, e.g., “The Handbook—A Guide to Fluorescent Probes and Labeling Technologies,” Tenth Edition, available on the internet at probes (dot) invitrogen (dot) com/handbook. The fluorescent label can be one or more of FAM, dRHO, 5-FAM, 6FAM, dR6G, JOE, HEX, VIC, TET, dTAMRA, TAMRA, NED, dROX, PET, BHQ, Gold540 and LIZ.

Using conventional techniques, the L-nucleic acid aptamers can be directly or indirectly labeled, e.g., the label is attached to the aptamer through biotin-streptavidin (e.g., synthesize a biotinylated aptamer, which is then capable of binding a streptavidin molecule that is itself conjugated to a detectable label; non-limiting example is streptavidin, phycoerythrin conjugated (SAPE)). Methods for chemical coupling using multiple step procedures include biotinylation, coupling of trinitrophenol (TNP) or digoxigenin using for example succinimide esters of these compounds. Biotinylation can be accomplished by, for example, the use of D-biotinyl-N-hydroxysuccinimide. Succinimide groups react effectively with amino groups at pH values above 7, and preferentially between about pH 8.0 and about pH 8.5. Alternatively, an aptamer is not labeled, but is later contacted with a second antibody that is labeled after the first antibody is bound to an antigen of interest.

Various enzyme-substrate labels may also be used in conjunction with L-nucleic acid aptamers. Such enzyme-substrate labels are available commercially (e.g., U.S. Pat. No. 4,275,149). The enzyme generally catalyzes a chemical alteration of a chromogenic substrate that can be measured using various techniques. For example, the enzyme may catalyze a color change in a substrate, which can be measured spectrophotometrically. Alternatively, the enzyme may alter the fluorescence or chemiluminescence of the substrate. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRP), alkaline phosphatase (AP),.beta.-galactosidase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of enzyme-substrate combinations include, but are not limited to, horseradish peroxidase (HRP) with hydrogen peroxidase as a substrate, wherein the hydrogen peroxidase oxidizes a dye precursor (e.g., orthophenylene diamine (OPD) or 3,3′,5,5′-tetramethylbenzidine hydrochloride (TMB)); alkaline phosphatase (AP) with para-nitrophenyl phosphate as chromogenic substrate; and.beta.-D-galactosidase.beta.-D-Gal) with a chromogenic substrate (e.g., p-nitrophenyl-.beta.-D-galactosidase) or fluorogenic substrate 4-methylumbelliferyl-p-D-galactosidase.

The L-nucleic acid aptamer(s) can be linked to a substrate such as a planar substrate. A planar array generally contains addressable locations (e.g., pads, addresses, or micro-locations) of biomolecules in an array format. The size of the array will depend on the composition and end use of the array. Arrays can be made containing from 2 different molecules to many thousands. Generally, the array comprises from two to as many as 100,000 or more molecules, depending on the end use of the array and the method of manufacture. A microarray for use with the invention comprises at least one biomolecule that identifies or captures a biomarker present in a biosignature of interest, e.g., a microRNA or other biomolecule or vesicle that makes up the biosignature. In some arrays, multiple substrates are used, either of different or identical compositions. Accordingly, planar arrays may comprise a plurality of smaller substrates.

The present inventors have shown that use of calf intestinal phosphatase (CIP) following iodoethanol-cleavage of DNA fragments aids in the sequencing of oligonucleotides which comprise L-deoxynucleoside α-thiotriphosphates.

Thus, according to still another aspect of the present invention there is provided a method of sequencing purified L-DNA molecules comprising:
(a) treating a sample comprising purified L-DNA molecules with a phosphatase (e.g. CIP) under conditions that remove 3′-monophosphates from the L-DNA molecules; and
(b) subjecting the sample to phosphorothioate sequencing, thereby sequencing purified L-DNA molecules.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

Generally, the nomenclature used herein, and the laboratory procedures utilized in the present invention include molecular, biochemical, microbiological and recombinant DNA techniques. Such techniques are thoroughly explained in the literature. General references are provided throughout this document.

### MATERIALS

All the L-DNA oligos (Table 1A and Table 1B, herein below) were synthesized on the H-8 oligo synthesizer (K&A Laborgeracte, Germany). All the D-DNA oligos (Tables 1A-B, herein below) were ordered from Genewiz (Jiangsu, China). L-deoxynucleoside phosphoramidites were purchased from ChemGenes (MA, U.S.). Hexaethylene glycol spacer (Sp18) phosphoramide was purchased from Glen Research (VA, U.S.). Fluorescein (FAM) and cyanine 5 (Cy5) phosphoramides, as well as 4-(4-dimethyl-aminophenylazo)benzoic acid (DABCYL) and monophosphate controlled pore glass (CPG) were purchased from Ruibiotech (Beijing, China). All the D- and L-DNA oligos were purified by HPLC or denaturing PAGE prior to use. L-deoxynucleoside triphosphates (L-dNTPs) and L-deoxynucleoside α-thiotriphosphates (L-dNTPαSs) were synthesized from L-deoxynucleosides (ChemGenes, MA, U.S.) 1. D-dNTPαSs were purchased from TriLink Biotechnologies Inc. (CA, U.S.). L-Dpo4-5 m with an N-terminal His₆ tag was expressed in Escherichia coli strain BL21 and purified as described in the literature². The FastPfu Fly DNA polymerase was purchased from TransGen Biotech (Beijing, China). D-Dpo4-5 m was synthesized and folded according to the previously reported methods except that automated peptide synthesizers were used, and norleucine (Nle) was replaced by methionine (Met)^{2,3}. 2-iodoethanol was purchased from Aladdin Bio-Chem Technology Co., Ltd. (Shanghai, China). Native human β-thrombin and native bovine α-thrombin of plasma origin were purchased from Haematologic Technologies (VT, U.S.). Streptavidin, calf intestinal alkaline phosphatase (CIP), and DNase I were purchased from New England Biolabs (MA, U.S.). Human serum was purchased from ZhongKeChenYu Biotech (Beijing, China). Monoclonal primary antibody targeting native human thrombin and Alexa Fluor 647-labelled polyclonal secondary antibody were purchased from Abcam (U.K.). ExRed was purchased from Beijing Zoman Biotech (Beijing, China). NHS-activated magnetic beads and SYBR-Green II were purchased from Thermo Fisher Scientific (MA, U.S.). Benzoyl-Phe-Val-Arg-AMC (AMC, 7-amino-4-methylcoumarin) was purchased from Sigma-Aldrich (MO, U.S.).

TABLE 1A
DNA sequences

| Oligo | Sequence |
|---|---|
| D/L-DNA library | 5′-CGGATCCAGTTACGGANNNNNNNNN |
| | NNNNNNNNNNNNNNNNNNNNNTTCATTC |
| | AGTAAGCTTCGG-3′- |
| | SEQ ID NO: 1 |
| D/L-DNA forward | 5′-CGGATCCAGTTACGGA-3′- |
| primer | SEQ ID NO: 2 |
| D/L-DNA reverse | 5′-CCGAAGCTTACTGAATGAA-3′- |
| primer | SEQ ID NO: 3 |
| D/L-DNA reverse | 5′-AAAAAAAAAAAAAAAAAAAA- |
| primer with Sp18 | Sp18-CCGAAGCTTACTGAATGA |
| | A-3′-SEQ ID NO: 4 and |
| | SEQ ID NO: 41 |
| D/L-DNA forward | 5′-CGCCCGCCGCGCCCCGCGCCC |
| primer with GC- | GTCCCGCCGCCCCCGCCCGCGGAT |
| clamp | CCAGTTACGGA-3′- |
| | SEQ ID NO: 5 |
| D/L-DNA forward | 5′-FAM-CGCCCGCCGCGCCCCGC |
| sequencing primer | GCCCGTCCCGCCGCCCCCGCCCGC |
| | GGATCCAGTTACGGA3′- |
| | SEQ ID NO: 6 |
| 3′-monophosphate | 5′-TGGATCCAGTTACGGA^{P}-3′- |
| labelled L-DNA | SEQ ID NO: 7 |
| D-6 aptamer | 5′-CGGATCCAGTTACGGACTGAAC |
| | AGAAGGGTGGTGTGGTTGGACTGTT |
| | CATTCAGTAAGCTTCGG-3′- |
| | SEQ ID NO: 8 |
| L-9-1 aptamer | 5′-CGGATCCAGTTACGGAACGCGT |
| | TTCAAGACTACCGTGTTTGTTCCGT |
| | TCATTCAGTAAGCTTCGG-3′- |
| | SEQ ID NO: 9 |
| L-9-1t aptamer | 5′-ACGGAACGCGTTTCAAGACTAC |
| | CGTGTTTGTTCCGT-3′- |
| | SEQ ID NO: 10 |
| Cy5-L-9-1t | 5′-Cy5-ACGGAACGCGTTTCAAGA |
| aptamer | CTACCGTGTTTGTTCCGT-3′- |
| | SEQ ID NO: 11 |
| L-9-1t-2 aptamer | 5′-GGAACGCGTTTCAAGACTACC |
| | GTGTTTGTTCC-3′- |
| | SEQ ID NO: 12 |
| L-9-2 aptamer | 5′-CGGATCCAGTTACGGATGAAC |
| | TTGTTGAAACCCAACGGGAGAATC |
| | GTTCATTCAGTAAGCTTCGG-3′- |
| | SEQ ID NO: 13 |
| L-9-2t aptamer | 5′-GATGAACTTGTTGAAACCCAA |
| | CGGGAGAATCGTTCATT-3′- |
| | SEQ ID NO: 14 |
| Cy5-L-9-2t aptamer | 5′-Cy5-GATGAACTTGTTGAAAC |
| | CCAACGGGAGAATCGTTCAT |
| | T-3′-SEQ ID NO: 15 |
| L-9-2-2t aptamer | 5′-GATGAACTTGTTGAAACCCA |
| | GCGGGAGATCGTTCAT |
| | T-3′-SEQ ID NO: 16 |
| D-DNA fluorophore | 5′-FAM-TCCGTAACTGGATCC |
| strand | G-3′-SEQ ID NO: 17 |
| L-DNA fluorophore | 5′-FAM-ACTGGATCCGAGCT |
| strand | G-3′-SEQ ID NO: 18 |
| D-DNA quencher | 5′-ACCCTTCTGTTCA- |
| strand | DABCYL-3′- |
| | SEQ ID NO: 19 |
| L-DNA quencher | 5′-ACGCGTTCCGT- |
| strand | DABCYL-3′- |
| | SEQ ID NO: 20 |
| D-DNA aptamer | 5′-CGGATCCAGTTACGGACTGAA |
| strand | CAGAAGGGTGGTGTGGTTGGACTG |
| | TTCA-3′-SEQ ID NO: 21 |
| L-DNA aptamer | 5′-CAGCTCGGATCCAGTTACGGA |
| strand | ACGCGTTTCAAGACTACCGTGTTT |
| | GTTCCGT-3′-SEQ ID NO: 22 |

TABLE 1B
L-DNA sequences for re-selection from a
partially randomized L-DNA library

| | |
|---|---|
| Partially randomized | 5′-CGGATCCAGTTACGGAtga |
| L-DNA library | acttgttgaaacccaacgggag |
| | aatcgttcaTTCAGTAAGCTTC |
| | GGTGG-3′-SEQ ID NO: 23 |
| L-DNA forward primer | 5′-CGGATCCAGTTACGGA- |
| | 3′-SEQ ID NO: 24 |
| L-DNA reverse primer | 5′-CCACCGAAGCTTACTGAA- |
| | 3′-SEQ ID NO: 25 |
| L-DNA reverse primer | 5′-AAAAAAAAAAAAAAAAAAAA- |
| with Sp18 | CSp18-CACCGAAGCTTACTGAA- |
| | 3′-SEQ ID NO: 26 and |
| | SEQ ID NO: 42 |
| L-13 aptamer | 5′-CGGATCCAGTTACGGATG |
| | AACTTGTTGAAACCCAACGGG |
| | AGCCTCGTTCATTCAGTAAGC |
| | TTCGGTGG-3′- |
| | SEQ ID NO: 27 |
| L-13t aptamer | 5′-GATGAACTTGTTGAAACCCA |
| | ACGGGAGCCTCGTTCATT- |
| | 3′-SEQ ID NO: 28 |
| Cy5-L-13t aptamer | 5′-Cy5-GATGAACTTGTTGAAAC |
| | CCAACGGGAGCCTCGTTCATT-3′ |
| | -SEQ ID NO: 29 |

### L-DNA Library Preparation

The 30 nt randomized region of D- or L-DNA library with 65 nt in total length was synthesized with molar concentration ratios of D- or L-dA, dC, dG, dT phosphoramidites of 1.5:1.25:1.15:1 to achieve approximately equal coupling efficiencies⁴. Native polyacrylamide gel electrophoresis (PAGE) purification was performed to remove the aggregation-prone DNA as described in the literature⁵. Briefly, 5 nmol of the synthetic D- or L-DNA library was loaded on slabs of 1 mm×200 mm×550 mm, separated by PAGE composed of a denaturing top section (1 mm×200 mm×50 mm) containing 7 M urea, 8% acrylamide in 0.5× Tris-borate-EDTA (TBE), and a nondenaturing bottom section (1 mm×200 mm×500 mm) containing 10% acrylamide, 10 mM Mg (OAc) 2 in 0.5× TBE. The gel was run at 10 W (constant power) for 6 h and stained by SYBR-Green II. The fastest-migrating ⅓ of the band was isolated and purified by the ‘crush and soak’ method⁶. Approximately 165 pmol of the native-PAGE-purified library (with ˜1×10¹⁴ distinct sequences) was amplified by natural or mirror-image PCR using L- or D-Dpo4-5 m with D- or L-DNA primers listed in Table 1A, herein above, in which the reverse primer contained a poly d (A) 20 tail modified by Sp18 to generate PCR product with strands of different lengths for strand separation by denaturing PAGE7. The natural and mirror-image PCR program settings were 86° C. for 3 min (initial denaturation); 86° C. for 30 sec, 50° C. for 1 min, and 65° C. for 2 min, for 15 cycles; 65° C. for 5 min (final extension). The 65 nt forward strand was separated from the 85 nt Sp18-modified reverse strand by 10% denaturing PAGE in 7 M urea and used as the starting D- or L-DNA library for aptamer selection.

### Selection of D- or L-DNA Aptamers Targeting Native Human Thrombin

Magnetic beads coupled with native human thrombin were prepared from N-hydroxy-succinimide (NHS)-activated magnetic beads according to the manufacturer's instructions (Thermo Fisher Scientific, MA, U.S.). Briefly, 300 μl of native human thrombin at a concentration of 0.1 mg/ml was mixed with 3 mg of NHS-activated magnetic beads in coupling buffer (20 mM HEPES-NaOH, 150 mM NaCl, 5% glycerol, pH 7.4). The coupling reaction was performed at room temperature for 2 h, before being quenched by 3 M ethanolamine at pH 9.0. After coupling, the beads were resuspended in 300 μl of selection buffer (20 mM HEPES-NaOH, 150 mM NaCl, 5 mM KCl, 2 mM MgCl₂, 1 mM CaCl₂), 0.05% (v/v) Tween-20, pH 7.4). For round 1 (R1), ˜600 μmol (˜3.6×10¹⁴ molecules with ˜1×10¹⁴ distinct sequences) of the D- or L-DNA library in a 250 μl volume was heated to 85° C. for 5 min in selection buffer and slowly cooled to 25° C. over 10 min, after which 50 μl protein-free NHS-activated magnetic beads were added and the mixture was incubated under gentle rotation at room temperature for 1 h. In each selection round, a negative selection step against 50 μl protein-free NHS-activated magnetic beads was performed. The supernatant was mixed with 100 μl magnetic beads coupled with native human thrombin in a total volume of 400 μl, and incubated under gentle rotation at room temperature for 1 h, after which the beads were separated from the supernatant by a DynaMag-2 magnet (Thermo Fisher Scientific, MA, U.S.) and briefly washed three times (10 sec per wash) with 400 μl selection buffer. The bound DNA was eluted from the beads by 25 mM NaOH and 5 mM EDTA, and precipitated by ethanol. The recovered D- or L-DNA was used as template for natural or mirror-image PCR amplification by L- or D-Dpo4-5 m to generate the D- or L-DNA pool for the next round. The number of natural or mirror-image PCR cycles for each selection round was determined based on the result of 10 μl scale PCR. As shown in Tables 2 and 3, the amount of DNA pool gradually decreased from ˜600 pmol in R1 to ˜50 pmol in R6 (for D-DNA pools), and from ˜600 pmol in R1 to ˜30 pmol in R9 (for L-DNA pools), respectively. The volume of magnetic beads coupled with native human thrombin gradually decreased from 100 μl in R1 to 10 μl in R6 (for D-DNA pools), and from 100 μl in R1 to 3 μl in R9 (for L-DNA pools), respectively. The wash step gradually increased from three 10-sec washes in R1 to six 10-min washes in R6 (for D-DNA pools), and from three 10-sec washes in R1 to eight 10-min washes in R9 (for L-DNA pools), respectively.

TABLE 2
Conditions for D-DNA aptamer selection

| | Amount | Thrombin- | | | | Number |
|---|---|---|---|---|---|---|
| | of | coupled | Incu- | | Natural | of |
| | D-DNA | bead | bation | | PCR | natural |
| | pool | volume | volume | Wash | volume | PCR |
| Round | (pmol) | (μl) | (μl) | condition | (μl) | cycles |
| 1 | 600 | 100 | 400 | 10 sec × 3 | 1500 | 20 |
| 2 | 200 | 50 | 280 | 10 sec × 4 | 1500 | 25 |
| 3 | 200 | 50 | 250 | 5 min × 4 | 1500 | 30 |
| 4 | 100 | 20 | 200 | 5 min × 5 | 1000 | 30 |
| 5 | 100 | 20 | 200 | 10 min × 5 | 1000 | 20 |
| 6 | 50 | 10 | 130 | 10 min × 6 | 500 | 15 |

TABLE 3
Conditions for L-DNA aptamer selection

| | | | | | | Number |
|---|---|---|---|---|---|---|
| | Amount | Thrombin- | | | Mirror- | of |
| | of | coupled | Incu- | | image | mirror- |
| | L-DNA | bead | bation | | PCR | image |
| | pool | volume | volume | Wash | volume | PCR |
| Round | (pmol) | (μl) | (μl) | condition | (μl) | cycles |
| 1 | 600 | 100 | 400 | 10 sec × 3 | 2500 | 20 |
| 2 | 200 | 50 | 280 | 10 sec × 4 | 1500 | 20 |
| 3 | 200 | 50 | 250 | 5 min × 4 | 1500 | 30 |
| 4 | 100 | 20 | 200 | 5 min × 5 | 1000 | 30 |
| 5 | 100 | 20 | 200 | 5 min × 6 | 1000 | 30 |
| 6 | 50 | 10 | 130 | 7 min × 6 | 500 | 25 |
| 7 | 50 | 5 | 190 | 10 min × 6 | 500 | 15 |
| 8 | 30 | 5 | 170 | 10 min × 8 | 500 | 15 |
| 9 | 30 | 3 | 200 | 10 min × 8 | 500 | 10 |

### Electrophoretic Mobility Shift Assay (EMSA)

The D- or L-DNA pools, and D- or L-DNA aptamers were heated to 85° C. for 5 min in selection buffer and slowly cooled to 25° C. over 10 min, before being mixed with native human thrombin or streptavidin in selection buffer with 10% (v/v) glycerol. The mixtures were incubated at room temperature for 30 min, and analyzed by 8% native PAGE in 1× running buffer (20 mM HEPES-NaOH, 50 mM NaOAc, 5 mM KOAc, 2 mM Mg (OAc) 2, 1 mM CaCl₂), pH 7.4 (for the D-or L-DNA pools, the D-6, Cy5-L-9-1t, and Cy5-L-13t aptamers), or by 10% native PAGE in 1× running buffer with 5% (v/v) glycerol added to both the gel and running buffer (for the Cy5-L-9-2t aptamer). The gel was run at 150 V (constant voltage) for 1-2 h, stained by SYBR-Green II, and scanned by the Amersham Typhoon Biomolecular Imager (Cytiva, U.S.) operated under Cy2 mode (for D- or L-DNA pools and the D-6 aptamer) or Cy5 mode (for the Cy5-labelled L-DNA aptamers). Gel quantitation was performed by the ImageJ software, with the dissociation constant (K_{d}) calculated by fitting the fraction bound to the sigmoidal model using the KaleidaGraph software (Synergy Software, PA, U.S.).

### Denaturing Gradient Gel Electrophoresis (DGGE)

The D- or L-DNA pools, and D- or L-DNA aptamers were amplified by natural or mirror-image PCR using L- or D-Dpo4-5 m with D- or L-DNA primers listed in Table 1A, with the forward primer containing a GC-rich region (GC-clamp) to prevent the double-stranded PCR product from complete melting during DGGE⁸. The natural or mirror-image PCR products were purified by 3% sieving agarose gel electrophoresis and mixed with 2× loading buffer (100 mM Tris-HCl, 10 mM EDTA, 30% glycerol, pH 7.0), and separated by 7.5% polyacrylamide gel (for D-DNA pools) or 10% polyacrylamide gel (for L-DNA pools) composed of a linear denaturant gradient from 2.1 M urea, 12% (v/v) formamide (top) to 4.2 M urea and 24% (v/v) formamide (bottom) in 1× Tris-acetate-EDTA (TAE). The gel was run at 100 V at 60° C. (constant temperature) for 6 h (for D-DNA pools) or at 75 V at 60° C. for 13 h (for L-DNA pools). For DGGE isolation of D- or L-DNA aptamer sequences, 500 ng of natural or mirror-image PCR products were separated by DGGE, stained by SYBR-Green II, isolated by cutting the gel on a 254 nm ultra-violet transilluminator, and purified by the ‘crush and soak’ method⁶, and re-amplified by natural or mirror-image PCR using L- or D-Dpo4-5 m with D- or L-DNA primers listed in Table 1A. To rule out the incorrect sequences from the L-9-2 band sequencing result, natural versions of the eight most probable L-DNA aptamer sequences in band L-9-2 (D-L-9-2-1 to D-L-9-2-8, FIG. 6B and Table 4, herein below) were amplified by natural PCR using the FastPfu Fly DNA polymerase with D-DNA primers, separated by DGGE, stained by SYBR-Green II, and scanned by the Amersham Typhoon Biomolecular Imager operated under Cy2 mode. The melting temperatures (Tₘ) were calculated by OligoCalc using default parameters of the nearest-neighbor thermodynamic model⁹.

TABLE 4
D-DNA oligos for DGGE analysis with
calculated melting temperature (Tₘ)

| | | Tₘ |
|---|---|---|
| Oligo | Sequence | (° C.) |
| D-L-9- | 5′- | 75.70 |
| 2-1 | CGGATCCAGTTACGGATGAACTTGTTGAAACCC | |
| | AGCGGGAGATCGTTCATTCAGTAAGCTTCGG-3′- | |
| | SEQ ID NO: 30 | |
| D-L-9- | 5′- | 75.63 |
| 2-2 | CGGATCCAGTTACGGATGAACTTGTTGAAACCC | |
| | AGCGGGAGAATCGTTCATTCAGTAAGCTTCGG-3′ | |
| | -SEQ ID NO: 31 | |
| D-L-9- | 5′- | 75.36 |
| 2-3 | CGGATCCAGTTACGGATGAACTTGTTGAAACCC | |
| | AGCGGAAGATCGTTCATTCAGTAAGCTTCGG-3′- | |
| | SEQ ID NO: 32 | |
| D-L-9- | 5′- | 74.36 |
| 2-4 | CGGATCCAGTTACGGATGAACTTGTTGAAACCC | |
| | AACGGAAGAATCGTTCATTCAGTAAGCTTCGG-3′ | |
| | -SEQ ID NO: 33 | |
| D-L-9- | 5′- | 74.41 |
| 2-5 | CGGATCCAGTTACGGATGAACTTGTTGAAACCC | |
| | AACGGAAGATCGTTCATTCAGTAAGCTTCGG-3′- | |
| | SEQ ID NO: 34 | |
| D-L-9- | 5′- | 74.75 |
| 2-6 | CGGATCCAGTTACGGATGAACTTGTTGAAACCC | |
| | AACGGGAGATCGTTCATTCAGTAAGCTTCGG-3′ | |
| | -SEQ ID NO: 35 | |
| D-L-9- | 5′- | 74.69 |
| 2-7 | CGGATCCAGTTACGGATGAACTTGTTGAAACCC | |
| | AACGGGAGAATCGTTCATTCAGTAAGCTTCGG-3′ | |
| | -SEQ ID NO: 36 | |
| D-L-9- | 5′- | 75.30 |
| 2-8 | CGGATCCAGTTACGGATGAACTTGTTGAAACCC | |
| | AGCGGAAGAATCGTTCATTCAGTAAGCTTCGG-3′ | |
| | -SEQ ID NO: 37 | |

### High-Throughput Sequencing of the Selected D-DNA Aptamers

The R6 D-DNA pool and the D-6 band isolated by DGGE were amplified by natural PCR using L-Dpo4-5 m with D-DNA primers listed in Table 1A. The PCR products were purified by 2.5% agarose, and sequenced on the Illumina HiSeq system (Illumina, CA, U.S.). The raw Illumina reads were processed and sorted by abundance using the Galaxy server (www(dot)usegalaxy(dot)org).

### Matrix-Assisted Laser Desorption Ionization Time of Flight Mass Spectrometry (MALDI-TOF MS)

MALDI-TOF MS was used to analyze the dephosphorylation of L-DNAs by CIP. Approximately 100 ng of 3′-monophosphate-labelled L-DNA oligo (Table 1A) was treated with 20 units of CIP, incubated in 1× CutSmart buffer (New England Biolabs, MA, U.S.) at 37° C. for 1 h, desalted by a C18 spin column (Thermo Fisher Scientific, MA, U.S.), and analyzed under positive linear mode by MALDI-TOF MS (Applied Biosystems 4800 plus, CA, U.S.).

### L-DNA Aptamer Sequencing

L-DNA aptamers isolated by DGGE were amplified by mirror-image PCR using D-Dpo4-5 m in 4 separate PCR reactions, within which one of the L-dNTPs was replaced by the corresponding L-dNTPαs10, using the 5′-FAM-labelled forward sequencing primer and unlabelled reverse primer listed in Table 1A. The 5′-FAM-labelled PCR products were purified by 10% denaturing PAGE in 7 M urea and dissolved in 10 mM Tris-HCl at pH 7.4 to a final concentration of ˜20 ng/μl. For each sequencing reaction, 5 μl of 5′-FAM-labelled L-DNA was mixed with 5 μl of cleavage solution containing 2% (v/v) 2-iodoethanol in ddH₂O, followed by being heated to 95° C. for 3 min, and quickly placed on ice. For the removal of 3′-monophosphate from the 2-iodoethanol-cleaved DNA fragments, each sequencing reaction was treated with 5 units of CIP, incubated in 1× CutSmart buffer at 37° C. for 1 h, before being mixed with 10 μl of 2× loading buffer containing 95% formamide and 10 mM EDTA. The samples were loaded on slabs of 0.4 mm×340 mm×300 mm, and analyzed by 10% denaturing PAGE in 7 M urea according to the previously reported methods¹⁰.

Isothermal titration calorimetry (ITC)

Native human thrombin, native bovine thrombin, and streptavidin in storage buffer were dialyzed against physiological buffer (20 mM HEPES-NaOH, 150 mM NaCl, 5 mM KCl, 2 mM MgCl₂, 1 mM CaCl₂), pH 7.4) at 4° C. for 16 h. D- and L-DNA aptamers were equilibrated in physiological buffer by ultrafiltration, before being heated to 85° C. for 5 min and slowly cooled to 25° C. over 10 min. ITC was performed using the MicroCal iTC₂₀₀ Microcalorimeter (GE Healthcare, U.K.) with 7 μM to 20 μM of native human thrombin, native bovine thrombin, or streptavidin in the reaction cell and 70 μM to 200 μM of D- or L-DNA aptamer in the injection syringe with stirring at 750 r.p.m. at 25° C. To measure the heat of dilution, 70 μM to 200 μM of D- or L-DNA aptamer was injected to physiological buffer in the absence of protein. Data fitting was performed using the MicroCal Origin software (GE Healthcare, U.K.).

### L-DNA Aptamer Sensor

The D- or L-DNA aptamer sensor containing 250 nM 5′-FAM-labelled fluorophore strand, 750 nM 3′-DABCYL-labelled quencher strand, and 500 nM of aptamer strand based on the D-6 or L-9-1t aptamer (Table 1A), was incubated with 300 nM native human thrombin in physiological buffer alone or physiological buffer with 10% (v/v) human scrum at 37° C. for 1 h or 4 h. Relative fluorescence was measured by the Varioskan Flash system (Thermo Fisher Scientific, MA, U.S.) with excitation wavelength at 494 nm and emission wavelength at 518 nm. The standard curve was plotted using 0, 125, 250, 500, or 1000 nM of native human thrombin and relative fluorescence was measured after incubation in physiological buffer at 37° C. for 1 h. Change of relative fluorescence unit (ARFU) over background (RFU measured with the D- or L-DNA aptamer sensor in physiological buffer alone) was used for data fitting. For measurements in physiological buffer with 10% (v/v) human serum, the standard curve was plotted using 0, 250, 500, 1000, or 2000 nM of native human thrombin and relative fluorescence was measured after incubation in physiological buffer with 10% human serum at 37° C. for 1 h. To evaluate the biostability of the D- and L-DNA aptamer sensors, the sensors were incubated in physiological buffer with 10% human scrum at 37° C. for up to 24 h (for the D-DNA aptamer sensor), or in physiological buffer with 83% (v/v) human serum at 37° C. for up to 24 h (for the D-DNA aptamer sensor) or up to 30 d (720 h) (for the L-DNA aptamer sensor). Samples were mixed with 2× loading buffer containing 95% formamide and 10 mM EDTA, and quickly placed at −20° C., before being analyzed by 10% denaturing PAGE in 7 M urea. Gel quantitation was performed by the ImageJ software, with the half-life (t_{1/2}) calculated by fitting the relative band intensity to the exponential decay model using the KaleidaGraph software (Synergy Software, PA, U.S.).

L-DNA aptamer Western blot

The Cy5-L-13t aptamer was heated to 85° C. for 5 min in physiological buffer, slowly cooled to 25° C. over 10 min. Native human thrombin was separated by 15% sodium dodecyl sulphate-polyacrylamide gel electrophoresis (SDS-PAGE) and transferred to a nitrocellulose membrane in 1× transfer buffer (25 mM Tris, 192 mM glycine, 20% (v/v) methanol, pH 8.3). The membrane was incubated in 1× blocking buffer (137 mM NaCl, 2.7 mM KCl, 10 mM Na₂HPO₄, 1.8 mM KH₂PO₄, 25 mg/ml bovine serum albumin, 0.05% (v/v) Tween-20, pH 7.4) at room temperature for 1 h, and incubated with 500 nM Cy5-L-13t aptamer in selection buffer at room temperature for 1 h. After the incubation, the membrane was washed five times (5 min per wash) with selection buffer and scanned by the Amersham Typhoon Biomolecular Imager operated under Cy5 mode. Traditional Western blot using the antibodies was performed according to the manufacturer's instructions (Abcam, U.K.).

### L-DNA Aptamer Enzymatic Inhibitor

L-DNA aptamers were heated to 85° C. for 5 min in physiological buffer, slowly cooled to 25° C. over 10 min, with native human thrombin added to a final concentration of 10 nM. The mixture was incubated in physiological buffer at room temperature for 30 min, followed by addition of 100 μM fluorogenic substrate benzoyl-Phe-Val-Arg-AMC. Relative fluorescence was measured by the Varioskan Flash system with excitation wavelength at 350 nm and emission wavelength at 450 nm. Relative thrombin enzymatic activity was determined with ARFU at 0 min set to 0 and ARFU of the negative control in physiological buffer alone set to 100%, and ARFU at 16 min was used to calculate the relative thrombin enzymatic activity. The half-maximum inhibitory concentration (IC₅₀) was calculated by fitting the relative thrombin enzymatic activity to the sigmoidal model using the KaleidaGraph software.

### L-DNA Aptamer Coagulation Assay

Human plasma was obtained from a healthy volunteer. The D- and L-DNA aptamers were heated to 85° C. for 5 min in 180 μl physiological buffer, slowly cooled to 25° C. over 10 min for annealing, and incubated with 180 μl of human plasma to a final concentration of 2.5 μM for the D- and L-DNA aptamers at room temperature for up to 10 min. The prothrombin time was measured by the Stago STA R Max automatic coagulant analyzer (Stago, France) according to the manufacturer's instructions.

### RESULTS

Validating and Optimizing a Selection Scheme for Identifying L-DNA Aptamers Directly from a Large Randomized L-DNA Library

Although Dpo4-5 m has been shown to amplify short DNA sequences efficiently^{7,8}, it has not been tested in the amplification of large randomized DNA libraries. Here, a large randomized D-DNA library of ˜1×10¹⁴ distinct sequences was prepared by solid-phase oligo synthesis, with 30 randomized nucleotides flanked by two constant regions for primer binding. The ability of L-Dpo4-5 m to amplify the large randomized D-DNA library was confirmed, and performed iterative rounds of selection for D-DNA aptamers targeting commercially available native human thrombin purified from plasma (Materials and Methods), against which high-affinity D-DNA aptamers have been previously selected^{30,31}. The progress of selection was monitored by electrophoretic mobility shift assay (EMSA), which accesses the overall binding fraction of the sequence pool during each selection round³². After 6 rounds of selection, ˜70% of the D-DNA pool bound 1 μM native human thrombin, but not 1 μM streptavidin. Next, round 6 (R6) D-DNA pools were sequenced by high-throughput sequencing, which revealed enrichment of multiple DNA sequences, although the most abundant sequence only accounted for ˜1.1% of the total reads.

In order to ascertain whether D-DNA sequences of similar lengths could be separated based on the different melting temperatures, DGGE was carried out to analyze the natural PCR products from R4 to R6, along with that from R0 prior to selection. While no clear band was observed in R0 and R4, single bands began to emerge in R5, with both the number and intensity of the bands increased in R6. Next, a single band (D-6) was isolated from R6, which accounted for ˜1.7% of the total lane fluorescence intensity of R6. The band D-6 was amplified by natural PCR using L-Dpo4-5 m with D-DNA primers and the PCR product was analyzed by another DGGE, which revealed a predominant band accounting for ˜35% of the total lane fluorescence intensity. The band was recovered from DGGE and its composition was analyzed by high-throughput sequencing, which revealed a single sequence accounting for ˜45% of the total reads (249272 in 554081 reads). In fact, the same sequence (D-6) was also found in the R6 pool prior to DGGE separation, but only accounting for ˜0.8% (ranked 4th) of the R6 reads. Therefore, although the D-6 sequence was rather rare in the R6 pool (˜1.7% revealed by DGGE, and ˜0.8% by high-throughput sequencing, respectively), it became predominant after DGGE separation and PCR amplification by L-Dpo4-5 m (˜35% revealed by DGGE, and ˜45% by high-throughput sequencing).

Next, band D-6 was sequenced using the phosphorothioate approach with D-deoxynucleoside α-thiotriphosphates (D-dNTPαS) and cleavage by 2-iodoethanol³³, which was recently adopted for L-DNA sequencing-by-synthesis¹³. The sequencing result was rather ambiguous due to band doubling, a phenomenon that was primarily attributed to the presence of 3′-hydroxyl and 3′-monophosphate groups among the cleaved DNA fragments³⁴. To address this issue, the 2-iodoethanol-cleaved DNA fragments were treated with calf intestinal alkaline phosphatase (CIP). Most of the band doubling disappeared after CIP treatment, likely due to the removal of 3′-monophosphates from the cleaved DNA fragments, and hence the sequence of band D-6 was readily determined. Prediction of secondary structure of the D-6 aptamer by Mfold³⁵ revealed that it contains a loop region that fits into the consensus sequence of previously identified D-DNA aptamers targeting native human thrombin³⁰. Finally, the D-DNA aptamer D-6 was prepared by solid-phase oligo synthesis, which bound native human thrombin with a dissociation constant (K_{d}) of 27 nM, as determined by isothermal titration calorimetry (ITC) in physiological buffer (20 mM HEPES-NaOH, 150 mM NaCl, 5 mM KCl, 2 mM MgCl₂, 1 mM CaCl₂), pH 7.4). Furthermore, the D-6 aptamer formed stable complexes with native human thrombin as revealed by EMSA, which was, as expected, digestible by DNase I.

### Mirror-Image Selection of L-DNA Aptamers Targeting Native Human Thrombin

A large randomized L-DNA library of ˜1×10¹⁴ distinct sequences was prepared by solid-phase oligo synthesis, with 30 randomized nucleotides flanked by two constant regions for primer binding, as with the D-DNA library. The L-DNA library was amplified by mirror-image PCR using D-Dpo4-5 m with L-DNA primers. As with the natural system, the progress of mirror-image selection was monitored by EMSA (FIG. 2A). After 9 rounds of selection, ˜70% of the L-DNA pool bound 1 μM native human thrombin, but not 1 μM streptavidin (FIGS. 2A, B). DGGE was carried out to analyze the mirror-image PCR products from R5 to R9, along with that from R0 prior to selection (FIG. 2C). While no clear band was observed in R0 and R5, single bands began to emerge in R6, with both the number and intensity of the bands increased from R7 to R9 (FIG. 2C). Two bands (L-9-1 and L-9-2) were isolated from R9, which accounted for ˜1.7% and ˜ 1.6% of the total lane fluorescence intensity of R9, respectively (FIG. 2C). The bands were amplified by mirror-image PCR using D-Dpo4-5 m with L-DNA primers in two separate reactions, and the mirror-image PCR products were analyzed by another DGGE, both revealing a predominant band, which accounted for ˜18% and ˜12% of the corresponding total lane fluorescence intensity, respectively (FIG. 2C).

In order to sequence the enriched L-DNA aptamers, band L-9-1 was isolated for L-DNA sequencing-by-synthesis using the phosphorothioate approach with L-deoxynucleoside α-thiotriphosphates (L-dNTPαS) and cleavage by 2-iodoethanol¹³. The sequencing result was again ambiguous due to band doubling, similar to the phosphorothioate sequencing results in the natural system (FIG. 5A). The 2-iodoethanol-cleaved L-DNA fragments were with CIP, and unexpectedly, it was found that the CIP treatment substantially improved the L-DNA sequencing results (FIG. 5B), likely through removal of 3′-monophosphates in L-DNAs through a previously unreported cross-chiral dephosphorylation activity of CIP. Hence, the sequence of band L-9-1 was readily determined.

Additionally, band L-9-2 was also sequenced using the phosphorothioate approach and it was observed that even with treatment by CIP, three nucleotide positions in the central region of the sequenced aptamer caused ambiguous reading (likely due to contaminating sequences) and resulted in eight most probable L-DNA aptamer sequences (FIG. 5C, and Table 4). It was reasoned that the incorrect sequences could be ruled out using DGGE by comparing the migration of potential aptamer sequences, since the correct sequence(s) should co-migrate with band L-9-2 for the identical melting temperature (FIG. 6A). Thus, natural versions (to save costs and the mirror-image enzymes) of the eight most probable L-DNA aptamer sequences in band L-9-2 (D-L-9-2-1 to D-L-9-2-8, Table 4) were screened by DGGE, in order to rule out the incorrect sequences. It was observed that only the D-L-9-2-7 sequence co-migrated with band L-9-2 FIG. 6B), suggesting that D-L-9-2-7 and band L-9-2 likely share the same sequence. Hence, the sequence of band L-9-2 was determined through a combination of a first DGGE to isolate (FIG. 2C), L-DNA sequencing-by-synthesis using the phosphorothioate approach, and a second DGGE to rule out the incorrect sequences (FIG. 6B).

### Characterizing the Selected L-DNA Aptamers

To evaluate the binding affinity of the sequenced L-DNA aptamers with native human thrombin, the L-DNA aptamer L-9-1 was prepared by solid-phase oligo synthesis (FIG. 3A), which bound native human thrombin with a K_{d} of 29 nM as determined by ITC in physiological buffer (FIG. 3B), comparable to that of the D-DNA aptamer D-6 (27 nM). The L-9-1 aptamer was truncated from 65 nt to 36 nt based on its secondary structure predicted by Mfold (FIG. 3C), and observed that the truncated aptamer (L-9-1t) bound native human thrombin with only slightly reduced affinity (K_{d}=39 nM, FIG. 3D). Meanwhile, binding was not detected between the L-9-1t aptamer with streptavidin, and the natural version of the L-9-1t (D-L-9-1t) aptamer with native human thrombin, suggesting that the binding between the L-9-1t aptamer and native human thrombin was both target- and chiral-specific. Further shortening the L-9-1t aptamer from 36 nt to 32 nt by truncating part of a stem region led to ˜3-fold reduction of affinity (K_{d}=111 nM, likely due to destabilization of the aptamer secondary structure. Furthermore, the 5′-cyanine 5 (Cy5)-labelled L-9-1t (Cy5-L-9-1t) aptamer formed stable complexes with native human thrombin with a K_{d} of 21 nM as determined by EMSA, and was, as expected, resistant to DNase I digestion (FIGS. 3E-G).

The L-DNA aptamer L-9-2 was also prepared by solid-phase oligo synthesis (FIG. 3H), which bound native human thrombin with a K_{d} of 168 nM as determined by ITC in physiological buffer (FIG. 3I). The L-9-2 aptamer was then truncated from 65 nt to 38 nt based on its secondary structure predicted by Mfold (FIG. 3J), and it was observed that the truncated aptamer (L-9-2t) bound native human thrombin with only slightly reduced affinity (K_{d}=251 nM, FIG. 3K). Meanwhile, binding was not detected between the L-9-2t aptamer with streptavidin, and the natural version of the L-9-2t (D-L-9-2t) aptamer with native human thrombin, suggesting that the binding between the L-9-2t aptamer and native human thrombin was both target- and chiral-specific. In addition, the 5′-Cy5-labelled L-9-2t (Cy5-L-9-2t) aptamer bound native human thrombin with a K_{d} of 355 nM as determined by EMSA (FIGS. 3L-N), and was, as expected, resistant to DNase I digestion (FIG. 3L). Furthermore, based on a DGGE-predicted contaminating sequence (D-L-9-2-1) from R9, a truncated version of the L-9-2-1 (L-9-2-1t) aptamer was prepared by solid-phase oligo synthesis, and it was observed that the binding affinity of the L-9-2-1t aptamer with native human thrombin was ˜5-fold lower than that of the L-9-2t aptamer (K_{d}=1337 nM).

To further evaluate the target-specificity of the L-DNA aptamers, the binding affinity of the L-9-1t and L-9-2t aptamers to native bovine thrombin was measured. Bovine thrombin exhibits ˜85% sequence identity with native human thrombin³⁸. It was observed that the L-9-1t and L-9-2t aptamers bound native bovine thrombin with K_{d} of 1027 nM and 426 nM, respectively, exhibiting ˜26-fold and ˜1.7-fold reduction in binding affinity compared with native human thrombin (39 nM and 251 nM, respectively). These results suggest that the L-9-1t aptamer binds native human much tighter than with native bovine thrombin, while the L-9-2t aptamer binds both with similar affinities.

### L-DNA Aptamer Sensor

To demonstrate the potential practical applications of the thrombin-binding L-DNA aptamers, a structure-switching L-DNA aptamer sensor was synthesized by combining the high-affinity thrombin-binding L-DNA aptamer L-9-1t with an L-DNA fluorophore strand with 5′-labelled fluorescein (FAM), and an L-DNA quencher strand with 3′-labelled 4-(4-dimethyl-aminophenylazo)benzoic acid (DABCYL), both hybridizing with the L-9-1t aptamer to form stable L-DNA duplexes³⁹ (FIG. 4A). Upon binding native human thrombin, the L-9-1t aptamer undergoes structure switching, releasing the quencher strand and leading to increases of relative fluorescence with linear response in the range of ˜ 125-1000 nM (FIG. 4B). In contrast, the L-DNA aptamer sensor did not respond to the addition of 1 μM streptavidin or 1 μM native bovine thrombin, consistent with the ITC results.

To evaluate the influence of serum enzymes on the biostability and thrombin-sensing ability of L-DNA aptamer sensor, the L-DNA aptamer sensor was incubated in physiological buffer with 10% (v/v) human serum, which provided a physiologically relevant nuclease-rich environment. The L-DNA aptamer sensor responded to the addition of native human thrombin in physiological buffer with 10% human serum with linear response in the range of ˜250-2000 nM (FIG. 4B). In parallel, a natural structure-switching sensor was constructed based on the D-DNA aptamer D-6 (D-DNA aptamer sensor). Next, 300 nM (final concentration) native human thrombin was added into physiological buffer containing the D- or L-DNA aptamer sensor, with 10% human serum, or with 50 units/ml DNase I (one of the major nucleases in serum⁴¹). After incubation in physiological buffer with 10% human serum for 1 h, the D- and L-DNA aptamer sensors measured thrombin concentrations at 416±62 nM and 457±72 nM, respectively, similar to those measured in physiological buffer alone (334±59 nM and 299±12 nM, respectively, FIG. 4C). However, after incubation in physiological buffer with 10% human serum for 4 h, the D-DNA aptamer sensor measured a thrombin concentration at 784±91 nM, whereas the L-DNA aptamer sensor measured a thrombin concentration at 375±54 nM (FIG. 4C). Moreover, after incubation in physiological buffer with 50 units/ml DNase I for 1 h and 4 h, the D-DNA aptamer sensor measured thrombin concentrations at 1219±57 nM and 984±52 nM, respectively, whereas the L-DNA aptamer sensor measured thrombin concentrations at 334±58 nM and 251±34 nM, respectively (FIG. 4C).

The error-prone measurements by the D-DNA aptamer sensor but not L-DNA aptamer sensor may be attributed to the increases of relative fluorescence resulting from degradation of the D-DNA aptamer sensor by serum enzymes or DNase I, causing premature release of the FAM fluorophore and DABCYL quencher, which was largely consistent with the estimated half-life (t_{1/2}) of ˜1.7 h for the D-DNA aptamer sensor incubated in physiological buffer with 10% human serum, as determined by denaturing polyacrylamide gel electrophoresis (PAGE). To further validate the biostability of the L-DNA aptamer sensor, the sensor was incubated in physiological buffer with 83% human serum, and no significant degradation of the L-DNA aptamer sensor was observed by denaturing PAGE after up to 30 d (720 h) of incubation, whereas the D-DNA aptamer sensor was rapidly degraded with an estimated tin of ˜2.1 h, largely consistent with the results from previous studies with other D-DNA aptamers in human scrum 3.42

L-DNA aptamer Western blot

To further explore the potential practical applications of the thrombin-binding L-DNA aptamers, the L-13t aptamer (which was selected and optimized in the section “Re-selection and optimization of L-DNA aptamers from a partially randomized L-DNA library” described below) was applied to a proof-of-concept Western blot experiment based on L-DNA aptamer for detecting native human thrombin immobilized on a nitrocellulose membrane (FIG. 4D). 6 ng to 180 ng of native human thrombin was analyzed by sodium dodecyl sulphate-polyacrylamide gel electrophoresis (SDS-PAGE), which was subsequently transferred to a nitrocellulose membrane and incubated with 500 nM Cy5-L-13t aptamer at room temperature for 1 h (FIG. 4E). Fluorescent bands consistent with the molecular mass of native human thrombin (˜36 kDa) were detected with a detection limit of <6 ng (FIG. 4F). In a control experiment, we analyzed 6 ng to 180 ng streptavidin by SDS-PAGE, transferred to a nitrocellulose membrane, and incubated with 500 nM Cy5-L-13t aptamer, with no clear band observed at the expected molecular mass of streptavidin (˜18 kDa) (FIG. 4F). In comparison, 6 ng to 180 ng native human thrombin was analyzed by traditional Western blot using a mouse monoclonal primary antibody targeting native human thrombin and an Alexa Fluor 647-labelled (with similar excitation and emission wavelengths as Cy5) goat anti-mouse IgG polyclonal secondary antibody, and detected fluorescent bands consistent with the molecular mass of native human thrombin (˜36 kDa) (FIG. 4G).

### L-DNA Aptamer Enzymatic Inhibitor

Next, the inhibition of thrombin enzymatic activity was tested by the thrombin-binding L-DNA aptamers L-9-1 and L-9-2 in physiological buffer with 100 μM benzoyl-Phe-Val-Arg-7-amino-4-methylcoumarin .(AMC) (FIG. 4H): a fluorogenic substrate for thrombin⁴⁴. The L-9-2 aptamer inhibited thrombin enzymatic activity with a half-maximum inhibitory concentration (IC₅₀) measured at 317±128 nM (FIG. 41), largely consistent with its K_{d} determined by ITC (168 nM, FIG. 3I). In comparison, the R0 L-DNA pool prior to selection did not inhibit thrombin enzymatic activity at concentrations of up to 8 μM. The inhibition of thrombin enzymatic activity by the truncated aptamer (L-9-2t) was also measured. A slightly higher IC₅₀ at 479±65 nM (FIG. 4I) was noted, largely consistent with its K_{d} determined by ITC (251 nM, FIG. 3K). However, the L-9-1 and L-9-1t aptamers did not inhibit thrombin enzymatic activity at concentrations of up to 8 μM, despite their higher binding affinity (K_{d}=29 nM and 39 nM, respectively), suggesting different binding sites of native human thrombin targeted by the L-9-1 and L-9-2 aptamers. In addition, the inhibition of thrombin enzymatic activity by the L-9-2t aptamer was shown to be chiral-specific in that the natural version of the L-9-2t aptamer (D-L-9-2t) did not inhibit thrombin enzymatic activity at concentrations of up to 8 μM.

### Re-Selection and Optimization of L-DNA Aptamers from a Partially Randomized L-DNA Library

The suboptimal binding of the L-9-2 aptamer with native human thrombin (with K_{d} measured at 168 nM) and inhibition of thrombin enzymatic activity (with IC₅₀ measured at 317±128 nM) prompted further improvement and optimization of the L-DNA aptamer for both binding and inhibitory characteristics.

For the re-selection and optimization of the L-9-2 aptamer, a partially randomized L-DNA library (R10) of ˜1×10¹¹ distinct sequences was synthesized by solid-phase oligo synthesis, with partial randomization of 34 nucleotides at a frequency of 10% based on the L-9-2 aptamer, flanked by two constant regions for primer binding. Next, mirror-image selection of the partially randomized L-DNA library targeting native human thrombin was performed (FIG. 7A). After 3 rounds of enrichment and mirror-image PCR amplification (FIGS. 7B,C), DGGE was applied to isolate a single band (L-13) from R13, which accounted for ˜0.2% of the total lane fluorescence intensity of R13 (FIG. 7D). Band L-13 was amplified by mirror-image PCR using D-Dpo4-5 m with L-DNA primers and the mirror-image PCR products was analyzed by another DGGE, revealing a predominant band which accounted for ˜13% of the corresponding total lane fluorescence intensity (FIG. 7D). L-DNA sequencing-by-synthesis was carried out using the phosphorothioate approach to determine the enriched L-DNA aptamer sequence, and a mutant sequence of the L-9-2 aptamer was identified with two adenosines mutated to cytosines in the partially randomized region (FIG. 7E). This re-selected L-DNA aptamer (L-13) bound native human thrombin with a K_{d} of 22 nM as determined by ITC in physiological buffer (FIG. 7F,G), displaying ˜8-fold improvement of binding affinity with native human thrombin compared with its parent aptamer L-9-2. The L-13 aptamer was truncated from 68 nt to 38 nt based on its secondary structure predicted by Mfold (FIG. 7H), and the truncated aptamer (L-13t) bound native human thrombin with only slightly reduced affinity (K_{d}=34 nM, FIG. 7I). Additionally, the 5′-Cy5-labelled L-13t (Cy5-L-13t) aptamer was found to form stable complexes with native human thrombin with a K_{d} of 28 nM as determined by EMSA (FIGS. 7J, K).

The inhibition of thrombin enzymatic activity by the re-selected L-13 and L-13t aptamers was tested in physiological buffer with 100 μM benzoyl-Phe-Val-Arg-AMC (FIG. 7L). The L-13 aptamer inhibited thrombin enzymatic activity with an IC₅₀ measured at 27±3 nM (FIG. 7M), largely consistent with its K_{d} determined by ITC (22 nM, FIG. 7G), displaying ˜12-fold improvement of inhibition of thrombin enzymatic activity compared with its parent aptamer L-9-2. In comparison, the R10 partially randomized L-DNA pool prior to re-selection did not inhibit thrombin enzymatic activity at concentrations of up to 1.4 μM. The inhibition of thrombin enzymatic activity by the truncated aptamer (L-13t) was also measured, and a slightly higher IC₅₀ at 46±4 nM (FIG. 7M) was observed, largely consistent with its K_{d} determined by ITC (34 nM, FIG. 7I).

As a final test for the selected L-DNA aptamers and a demonstration of their clinical potential, an in vitro coagulation assay on human plasma was carried out. On addition of 2.5 μM L-9-1t and L-13t aptamers, the prothrombin time was measured to be ˜4- and ˜2-fold longer than those of the controls without L-DNA aptamers, or the natural version of the L-9-1t (D-L-9-1t) aptamer, respectively (FIG. 7N).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

1 Nolte, A., Klussmann, S., Bald, R., Erdmann, V. A. & Furste, J. P. Mirror-design of L-oligonucleotide ligands binding to L-arginine. Nat. Biotechnol. 14, 1116-1119 (1996).
2 Klussmann, S., Nolte, A., Bald, R., Erdmann, V. A. & Furste, J. P. Mirror-image RNA that binds D-adenosine. Nat. Biotechnol. 14, 1112-1115 (1996).
3 Williams, K. P. et al. Bioactive and nuclease-resistant L-DNA ligand of vasopressin. Proc. Natl Acad. Sci. USA 94, 11285-11290 (1997).
4 Dunn, M. R., Jimenez, R. M. & Chaput, J. C. Analysis of aptamer discovery and technology. Nat. Rev. Chem. 1, 0076 (2017).
5 Zhou, J. & Rossi, J. Aptamers as targeted therapeutics: current potential and challenges. Nat. Rev. Drug Discov. 16, 181-202 (2017).
6 Wang, Z., Xu, W., Liu, L. & Zhu, T. F. A synthetic molecular system capable of mirror-image genetic replication and transcription. Nat. Chem. 8, 698-704 (2016).
7 Xu, W. et al. Total chemical synthesis of a thermostable enzyme capable of polymerase chain reaction. Cell Discov. 3, 17008 (2017).
8 Jiang, W. et al. Mirror-image polymerase chain reaction. Cell Discov. 3, 17037 (2017).
9 Liu, X. & Zhu, T. F. Sequencing mirror-image DNA chemically. Cell Chem. Biol. 25, 1151-1156 (2018).
10 Wang, M. et al. Mirror-image gene transcription and reverse transcription. Chem 5, 848-857 (2019).
11 Ling, J.-J. et al. Mirror-image 5S ribonucleoprotein complexes. Angew. Chem. Int. Ed. 59, 3724-3731 (2020)
12 Chen, J., Chen, M. & Zhu, T. F. Translating protein enzymes without aminoacyl-tRNA synthetases. Chem 7, 786-798 (2021).
13 Fan, C., Deng, Q. & Zhu, T. F. Bioorthogonal information storage in L-DNA with a high-fidelity mirror-image Pfu DNA polymerase. Nat. Biotechnol. 39:1548-1555 (2021).
14 Tuerk, C. & Gold, L. Systematic evolution of ligands by exponential enrichment: RNA ligands to bacteriophage T4 DNA polymerase. Science 249, 505-510 (1990).
15 Ellington, A. D. & Szostak, J. W. In vitro selection of RNA molecules that bind specific ligands. Nature 346, 818-822 (1990).
16 Wilson, D. S. & Szostak, J. W. In vitro selection of functional nucleic acids. Annu. Rev. Biochem. 68, 611-647 (1999).
17 Gawande, B. N. et al. Selection of DNA aptamers with two modified bases. Proc. Natl Acad. Sci. USA 114, 2898-2903 (2017).
18 Liu, Z. X., Chen, T. J. & Romesberg, F. E. Evolved polymerases facilitate selection of fully 2′-OMe-modified aptamers. Chem. Sci. 8, 8179-8182 (2017).
19 Dunn, M. R., McCloskey, C. M., Buckley, P., Rhea, K. & Chaput, J. C. Generating biologically stable TNA aptamers that function with high affinity and thermal stability. J. Am. Chem. Soc. 142, 7721-7724 (2020).
20 Pinheiro, V. B. et al. Synthetic genetic polymers capable of heredity and evolution. Science 336, 341-344 (2012).
21 Kimoto, M., Yamashige, R., Matsunaga, K.-i., Yokoyama, S. & Hirao, I. Generation of high-affinity DNA aptamers using an expanded genetic alphabet. Nat. Biotechnol. 31 (2013).
22 Hoshika, S. et al. Hachimoji DNA and RNA: A genetic system with eight building blocks. Science 363, 884-887 (2019).
23 Purschke, W. G., Eulberg, D., Buchner, K., Vonhoff, S. & Klussmann, S. An L-RNA-based aquaretic agent that inhibits vasopressin in vivo. Proc. Natl Acad. Sci. USA 103, 5173-5178 (2006).
24 Sczepanski, J. T. & Joyce, G. F. Binding of a structured D-RNA molecule by an L-RNA aptamer. J. Am. Chem. Soc. 135, 13290-13293 (2013).
25 Umar, M. I. & Kwok, C. K. Specific suppression of D-RNA G-quadruplex-protein interaction with an L-RNA aptamer. Nucleic Acids Res. 48, 10125-10141 (2020).
26 Olea Jr, C., Weidmann, J., Dawson, Philip E. & Joyce, Gerald F. An L-RNA aptamer that binds and inhibits RNase. Chem. Biol. 22, 1437-1441 (2015).
27 Yatime, L. et al. Structural basis for the targeting of complement anaphylatoxin C5a using a mixed L-RNA/L-DNA aptamer. Nat. Commun. 6, 6481 (2015).
28 Pech, A. et al. A thermostable D-polymerase for mirror-image PCR. Nucleic Acids Res. 45, 3997-4005 (2017).
29 Muyzer, G. & Smalla, K. Application of denaturing gradient gel electrophoresis (DGGE) and temperature gradient gel electrophoresis (TGGE) in microbial ecology. Antonie van Leeuwenhoek 73, 127-141 (1998).
30 Bock, L. C., Griffin, L. C., Latham, J. A., Vermaas, E. H. & Toole, J. J. Selection of single-stranded DNA molecules that bind and inhibit human thrombin. Nature 355, 564-566 (1992).
31 Tasset, D. M., Kubik, M. F. & Steiner, W. Oligonucleotide inhibitors of human thrombin that bind distinct epitopes. J. Mol. Biol. 272, 688-698 (1997).
32 Dobbelstein, M. & Shenk, T. In vitro selection of RNA ligands for the ribosomal L22 protein associated with Epstein-Barr virus-expressed RNA by using randomized and cDNA-derived RNA libraries. J. Virol. 69, 8027-8034 (1995).
33 Gish, G. & Eckstein, F. DNA and RNA sequence determination based on phosphorothioate chemistry. Science 240, 1520-1522 (1988).
34 Nakamaye, K. L., Gish, G., Eckstein, F. & Vosberg, H. P. Direct sequencing of polymerase chain-reaction amplified DNA fragments through the incorporation of deoxynucleoside alpha-thiotriphosphates. Nucleic Acids Res. 16, 9947-9959 (1988).
35 Zuker, M. Mfold web server for nucleic acid folding and hybridization prediction. Nucleic Acids Res. 31, 3406-3415 (2003).
36 DeAnda, A., Jr. et al. Pilot study of the efficacy of a thrombin inhibitor for use during cardiopulmonary bypass. Ann. Thorac. 58, 344-350 (1994).
37 Bode, W. et al. The refined 1.9 Å crystal structure of human alpha-thrombin: interaction with D-Phe-Pro-Arg chloromethylketone and significance of the Tyr-Pro-Pro-Trp insertion segment. EMBO J. 8, 3467-3475 (1989).
38 Liu, X. et al. RNA aptamers specific for bovine thrombin. J. Mol. Recognit. 16, 23-27 (2003).
39 Nutiu, R. & Li, Y. F. Structure-switching signaling aptamers. J. Am. Chem. Soc. 125, 4771-4778 (2003).
40 Li, L.-L., Ge, P., Selvin, P. R. & Lu, Y. Direct detection of adenosine in undiluted serum using a luminescent aptamer sensor attached to a terbium complex. Anal. Chem. 84, 7852-7856 (2012).
41 Barra, G. B. et al. EDTA-mediated inhibition of DNases protects circulating cell-free DNA from ex vivo degradation in blood samples. Clin. Biochem. 48, 976-981 (2015).
42 Kratschmer, C. & Levy, M. Effect of chemical modifications on aptamer stability in serum. Nucleic Acid Ther. 27, 335-344 (2017).
43 Wang, Y., Li, Z. & Yu, H. Aptamer-based Western blot for selective protein recognition. Front. Chem. 8, 870528 (2020)
44 Li, M.-L., Ren, Y.-J., Dong, M.-H. & Ren, W.-X. Design, synthesis and structural exploration of novel fluorinated dabigatran derivatives as direct thrombin inhibitors. Eur. J. Med. Chem. 96, 122-138 (2015).
45 Fang, X. H. & Tan, W. H. Aptamers generated from cell-SELEX for molecular medicine: a chemical biology approach. Acc. Chem. Res. 43, 48-57 (2010).
46 Li, S. et al. Identification of an aptamer targeting hnRNP A1 by tissue slide-based SELEX. J. Pathol. 218, 327-336 (2009).
47 Sczepanski, J. T. & Joyce, G. F. A cross-chiral RNA polymerase ribozyme. Nature 515, 440-442 (2014).
48 Scheitl, C. P. M., Ghaem Maghami, M., Lenz, A.-K. & Hobartner, C. Site-specific RNA methylation by a methyltransferase ribozyme. Nature 587, 663-667 (2020).
49 Chandrasekar, J. & Silverman, S. K. Catalytic DNA with phosphatase activity. Proc. Natl Acad. Sci. USA 110, 5315-5320 (2013).
50 Peplow, M. Mirror-image enzyme copies looking-glass DNA. Nature 533, 303-304 (2016).
51 Peplow, M. A Conversation with Ting Zhu. ACS Cent. Sci. 4, 783-784 (2018).
52 Mattheakis, L. C., Bhatt, R. R. & Dower, W. J. An in vitro polysome display system for identifying ligands from very large peptide libraries. Proc. Natl Acad. Sci. USA 91, 9022-9026 (1994).
53 Roberts, R. W. & Szostak, J. W. RNA-peptide fusions for the in vitro selection of peptides and proteins. Proc. Natl Acad. Sci. USA 94, 12297-12302 (1997).
54 Reuter, Jason A., Spacek, D. V. & Snyder, Michael P. High-throughput sequencing technologies. Mol. Cell 58, 586-597 (2015).
55 Iliuk, A. B., Hu, L. & Tao, W. A. Aptamer in bioanalytical applications. Anal. Chem. 83, 4440-4452 (2011).
56 Brumbt, A. et al. Chiral stationary phase based on a biostable L-RNA aptamer. Anal. Chem. 77, 1993-1998 (2005).
57 Idili, A., Parolo, C., Alvarez-Diduk, R. & Merkoçi, A. Rapid and efficient detection of the SARS-COV-2 spike protein using an electrochemical aptamer-based sensor. ACS sens. 6, 3093-3101 (2021).

What is claimed is:

1. A method for screening a plurality of L-nucleic acid aptamers for an L-nucleic acid aptamer having a binding affinity to a target molecule, comprising:
(a) contacting said plurality of L-nucleic acid aptamers with the target molecule under conditions that selectively capture target-bound L-nucleic acid aptamers from said plurality of L-nucleic acid aptamers;
(b) amplifying L-nucleic acid aptamers of said target-bound L-nucleic acid aptamers to generate amplified, double-stranded L-nucleic acid oligonucleotides; and
(c) isolating amplified double stranded L-nucleic acid oligonucleotides using an electrophoresis based method, thereby screening the plurality of L-nucleic acid aptamers.

2. The method of claim 1, further comprising converting amplified double-stranded L-nucleic oligonucleotides to single stranded oligonucleotides following step (b) and prior to step (c).

3. The method of claim 2, wherein steps (a) and (b) and said step of converting are repeated at least three times prior to said isolating in order to enrich for said target-bound L-nucleic acid aptamers.

4. The method of claim 3, further comprising monitoring enrichment of said target-bound L-nucleic acid aptamers.

5. The method of claim 4, wherein said monitoring is effected by an electrophoretic mobility shift assay (EMSA).

6. The method of claim 1, wherein said electrophoresis based method is selected from the group consisting of Native PAGE; Denaturing PAGE; Denaturing gradient gel electrophoresis (DGGE); Constant denaturing gel electrophoresis (CDGE) and Temporal temperature gradient gel electrophoresis (TTGE).

7. The method of claim 1, wherein said electrophoresis based method comprises DGGE.

8. The method of claim 1, wherein said target molecule is selected from the group consisting of a peptide, a polypeptide, a small molecule, a carbohydrate and a nucleic acid molecule.

9. The method of claim 1, wherein said target molecule is comprised in a cell or a tissue.

10. The method of claim 1, wherein said amplifying utilizes a D-amino acid polymerase.

11. The method of claim 10, wherein said D-amino acid polymerase is selected from the group consisting of D-ASFV pol X, D-Taq polymerase, D-Pfu polymerase, Sulfolobus and solfataricus P2 DNA polymerase IV (DPO4), a fusion protein comprising said DPO4 and a polymerase having an amino acid sequence at least 80% identical to said DPO4.

12. The method of claim 1, further comprising sequencing said isolated members following step (c) so as to obtain the sequence of the L-nucleic acid aptamer having a binding affinity to said target molecule.

13. The method of claim 12, wherein said sequencing is effected using a method selected from the group consisting of L-DNA chemical sequencing; L-DNA phosphorothioate sequencing; L-DNA dideoxy sequencing; L-DNA Ion Torrent sequencing; L-DNA Illumina sequencing; and L-DNA Nanopore sequencing.

14. The method of claim 13, wherein said method is L-DNA phosphorothioate sequencing.

15. The method of claim 14, further comprising contacting said amplified double stranded L-nucleic acid oligonucleotides with a phosphatase prior to said sequencing.

16. The method of claim 12, further comprising constructing an additional aptamer library, wherein each member of said library has an identical 5′ and 3′ nucleic acid sequence and is up to 60% randomized compared to the sequence of the isolated L-nucleic acid aptamer.

17. The method of claim 1, further comprising synthesizing said plurality of L-nucleic acid aptamers prior to step (a).

18. The method of claim 17, wherein said synthesizing comprises error-prone PCR.

19. A method of sequencing purified L-DNA molecules comprising:
(a) treating a sample comprising said purified L-DNA molecules with a phosphatase under conditions that remove 3′-monophosphates from said L-DNA molecules; and
(b) subjecting said sample to phosphorothioate sequencing, thereby sequencing purified L-DNA molecules.

20. An isolated thrombin-binding L-DNA aptamer comprising a sequence as set forth in SEQ ID NOs: 10, 12, 14, 16, 27 or 28 or a sequence at least 80% identical to said SEQ ID Nos: 10, 12, 14, 16, 27 or 28.
